(19) United States
(12) Patent Application Publication
(10) Pub. No.: US 2025/0270538 A1
(43) Pub. Date: Aug. 28, 2025

(54) METHODS FOR THE SEPARATION OF NUCLEIC ACIDS WITH SLALOM CHROMATOGRAPHY

(71) Applicant: Waters Technologies Corporation, Milford, MA (US)

(72) Inventors: Kevin Wyndham, Worcester, MA (US); Peng Chen, Lebanon, CT (US); Fabrice Gritti, Franklin, MA (US)

(73) Assignee: Waters Technologies Corporation, Milford, MA (US)

(21) Appl. No.: 19/067,433

(22) Filed: Feb. 28, 2025

Related U.S. Application Data
(60) Provisional application No. 63/701,042, filed on Sep. 30, 2024.
(60) Provisional application No. 63/559,084, filed on Feb. 28, 2024.

(51) Int. Cl. C12N 15/10; G01N 30/32; G01N 30/74; G01N 30/88

(52) U.S. Cl. CPC C12N 15/101; G01N 30/32; G01N 30/74; G01N 2030/324; G01N 2030/8827

(57) ABSTRACT

Disclosed herein are methods for the separation of nucleic acids using slalom chromatography. The provided methods afford robust separation of nucleic acid species, including DNA, RNA, and mixtures thereof. Further, the methods are suitable for the separation of similar nucleic acid species that differ by topological conformations. Separation of nucleic acid species using the disclosed chromatographic methods can be achieved in under 10 minutes (e.g., between 1 and 4 minutes) while maintaining high levels of efficiency and peak resolution.

### CROSS REFERENCE TO RELATED APPLICATIONS

This application claims priority to and benefit of U.S. provisional application No. 63/559,084, filed Feb. 28, 2024, and entitled “Methods for the Separation of Nucleic Acids with Slalom Chromatography.” This application also claims priority to and benefit of U.S. provisional application No. 63/701,042, filed Sep. 30, 2024, and entitled “Methods for the Separation of Nucleic Acids with Slalom Chromatography.” The contents of both U.S. provisional application Nos. 63/559,084 and 63/701,042 are incorporated herein by reference in their entireties.

### FIELD OF INVENTION

The present disclosure relates generally to methods of separating nucleic acids using liquid chromatography, particularly slalom chromatography.

### BACKGROUND

Nucleic acids form the basis of molecular cloning techniques, are critical in the manufacture of biologic therapeutics such as antibodies and are at the forefront of new modalities such as cell/gene therapies and RNA-based vaccines. As such, the ability to manipulate, separate, and characterize nucleic acids is foundational across academic, biotechnology, and pharmaceutical industries. Existing methods for the separation and characterization of nucleic acids are slow (often requiring 30+ minutes to multiple days) and lack the higher resolution that exists for the separation of other macromolecules, such as proteins. Gel electrophoresis, the predominant method for separating nucleic acids, requires sample manipulation for visualization (e.g., the addition of a dye such as ethidium bromide), optimization of the gel concentration based on the nucleic acid size, and hinders downstream processing of target nucleic acid species (e.g., extraction from the gel itself, which substantially lowers yield and introduces contaminants). As such, there is a need in the art for methods that can quickly separate nucleic acids with limited sample manipulation and high resolution.

### SUMMARY OF INVENTION

The ability to characterize nucleic acids robustly and efficiently is critical in the development of therapeutics across modalities, including but not limited to, antibodies, cell/gene therapies, and mRNA vaccines. The present technology provides for methods for column-based separation of nucleic acids in 10 minutes or less (e.g., 10 minutes, 9 minutes, 8 minutes, 6 minutes, 5 minutes, 3 minutes, 2 minutes, etc.).

Accordingly, in one aspect, disclosed herein is a method of separating DNA and RNA, the method comprising, a) loading a sample comprising a plurality of single-stranded RNA (ssRNA) molecules and a plurality of double-stranded DNA (dsDNA) molecules onto a chromatographic column, wherein the column comprises a plurality of porous or non-porous particles; b) eluting the sample from the column, wherein the eluting is performed in between 0.6 and 10 minutes; and c) detecting the plurality of ssRNA molecules and/or the plurality of dsDNA molecules in the eluent, wherein the plurality of ssRNA molecules form at least one distinct peak and wherein the plurality of dsDNA molecules form at least one distinct peak. In some embodiments, the eluting is performed in between 0.6 and 6 minutes. In some embodiments, the eluting is performed in between 1 and 3 minutes. In some embodiments, the eluting results in a zone retention factor of less than 0.35. In some embodiments, the sample further comprises a plurality of single-stranded DNA and/or a plurality of double-stranded RNA.

In one aspect, disclosed herein is a method of separating double-stranded DNA (dsDNA), the method comprising, a) loading a sample comprising a plurality of dsDNA molecules onto a chromatographic column, wherein the column comprises a plurality of porous or non-porous particles, b) eluting the sample from the column, wherein the eluting results in a zone retention factor (k1) of less than 0.35, and c) detecting the plurality of dsDNA molecules in the eluent. In some embodiments, the sample further comprises a plurality of single-stranded RNA (ssRNA) molecules, a plurality of double-stranded RNA (dsRNA) molecules, and/or a plurality of single-stranded DNA (ssDNA) molecules. In some embodiments, the dsDNA is linear, circular or supercoiled. In some embodiments, the ssRNA is linear or circular. In some embodiments, step c) further comprises detecting the plurality of ssRNA molecules, the plurality of dsRNA molecules, and/or the plurality of ssDNA molecules in the eluent. In some embodiments, eluting with a k₁ of less than 0.35 (e.g., 0.33 or less) can be controlled, tailored, or adjusted to a desired k₁ value by the selection of ionic strength conditions of eluting the sample from the column. During elution from a column, ionic strength conditions can be modified in numerous ways, for example, the salt concentration of the mobile phase can be increased or decreased. Alternatively or additionally, the surface of the stationary phase or materials within the flow path of the column can be modified to affect the net charge (e.g., increasing a net negative charge of a surface).

In one aspect, disclosed herein is a method of separating DNA, the method comprising a) loading a sample comprising a plurality of dsDNA molecules and ssDNA molecules onto a chromatographic column, wherein the column comprises a plurality of porous or non-porous particles, b) eluting the sample from the column, wherein the eluting results in a zone retention factor (k₁) of less than 0.35, and c) detecting the plurality of dsDNA molecules and ssDNA molecules in the eluent.

In one aspect, disclosed herein is a method of separating RNA, the method comprising a) loading a sample comprising a plurality of ssRNA molecules and a plurality of dsRNA molecules onto a chromatographic column, wherein the column comprises a plurality of porous or non-porous particles, b) eluting the sample from the column, wherein the eluting results in a zone retention factor (k₁) of less than 0.35, and c) detecting the plurality of ssRNA molecules and/or the plurality of dsRNA molecules in the eluent.

In one aspect, disclosed herein is a method of separating DNA and RNA, the method comprising a) loading a sample comprising a plurality of ssRNA molecules and a plurality of dsDNA molecules onto a chromatographic column, wherein the column comprises a plurality of porous or non-porous particles, b) eluting the sample from the column, wherein the eluting results in a zone retention factor (k₁) of less than 0.35, and c) detecting the plurality of ssRNA molecules and/or the plurality of dsDNA molecules in the eluent.

In some embodiments of the above methods, the eluting is performed in between 0.6 and 10 minutes. In some embodiments, the eluting is performed in between 0.6 and 6 minutes. In some embodiments, the eluting is performed in between 1 and 3 minutes. In some embodiments, step b) is performed with an efficiency (N)>40,000. In some embodiments, the eluent is collected.

In some embodiments, the dsDNA, the ssDNA, the ssRNA, and/or the dsRNA comprise between about 1,000 base pairs to about 100,000 base pairs.

In some embodiments, the plurality of particles are between about 1 μm to about 10 μm in diameter. In some embodiments, the particles are porous. In some embodiments, the plurality of particles have an average pore size of between about 100 Å to 900 Å. In some embodiments, the plurality of particles have an average pore size of about 125 Å. In some embodiments, the plurality of particles have an average pore size of about 250 Å. In some embodiments, the plurality of particles have a specific pore volume of between 0.5 to 1.3 cc/g.

In some embodiments, at least a portion of an interior surface of the chromatographic column body is coated with an alkylsilyl material. In some embodiments, the alkylsilyl material is a hydrophilic, non-ionic layer of polyethylene glycol silane. In some embodiments, the alkylsilyl material is a product of vapor deposition of precursors bis(trichlorosilyl)ethane or bis(trimethoxysilyl)ethane.

In some embodiments, the chromatographic column is connected to a high-performance liquid chromatography (HPLC) system or an ultra-high performance liquid chromatography (UHPLC) system. In some embodiments, step c) is performed using an ultraviolet (UV) detector or a tunable ultraviolet (TUV) detector. In some embodiments, step c) is performed using a multi-angle light scattering (MALS) detector.

In some embodiments, step b) is performed using a flow rate of greater than 0.5 mL/min and wherein the dsDNA, the ssDNA, the ssRNA, and/or the dsRNA comprise between about 2,000 base pairs to about 25,000 base pairs. In some embodiments, the plurality of porous or non-porous particles have a diameter of between 1.5-1.9 μm.

In some embodiments, step b) is performed using a flow rate of greater than 0.75 mL/min and wherein the dsDNA, the ssDNA, the ssRNA, and/or the dsRNA comprise between about 5,000 base pairs to about 50,000 base pairs. In some embodiments, the plurality of porous or non-porous particles have a diameter of between 2.8-3.2 μm.

In some embodiments, step b) is performed using a flow rate of greater than 1.0 mL/min and wherein the dsDNA, the ssDNA, the ssRNA, and/or the dsRNA comprise between about 25,000 base pairs to about 50,000 base pairs. In some embodiments, the plurality of porous or non-porous particles have a diameter of between 4.8-5.2 μm.

In some embodiments, step b) is performed using a flow rate of greater than 2.5 mL/min and wherein the dsDNA, the ssDNA, the ssRNA, and/or the dsRNA comprise between about 25,000 base pairs to about 100,000 base pairs. In some embodiments, the plurality of porous or non-porous particles have a diameter of between 9.8-10.2 μm.

### BRIEF DESCRIPTION OF THE DRAWINGS

The technology will be more fully understood from the following detailed description taking in conjunction with the accompanying drawings.

FIG. 1 is a graphical illustration of a method of separating nucleic acid molecules according to some embodiments of the present technology.

FIG. 2 provides a chromatograph of double-stranded DNA species separated according to some embodiments of the present technology.

FIG. 3 provides a chromatograph of circular and linear DNA species separated according to some embodiments of the present technology.

FIG. 4 provides a chromatograph of single-stranded and double-stranded RNA separated according to some embodiments of the present technology.

FIG. 5 provides chromatographs of double-stranded DNA separated using mobile phases of varying ionic strengths.

FIG. 6 provides chromatographs of double-stranded RNA separated at low and high shear flow rates.

FIG. 7A and FIG. 7B provide chromatographs of double-stranded DNA samples separated at various flow rates. FIG. 7A provides chromatographs of supercoiled dsDNA separated from linear dsDNA. FIG. 7B provides chromatographs of circular dsDNA separated from linear dsDNA.

FIG. 8A and FIG. 8B demonstrate the correlation between nucleic acid length and k coefficients of dsDNA separated using methods according to some embodiments of the present technology.

FIG. 9 provides a chromatograph of a DNA and RNA mixture separated according to embodiments of the present technology.

FIG. 10 provides a chromatograph of a DNA digest separated according to some embodiments of the technology.

FIG. 11 provides a chromatograph of a small interfering RNA (siRNA) calibrant separated according to some embodiments of the technology and detected using multi-angle light scattering (MALS).

FIGS. 12A-12F provide chromatographs of a DNA digest separated according to some embodiments of the technology and detected using multi-angle light scattering (MALS).

FIG. 12A provides the chromatograph before UV broadening.

FIG. 12B provides the chromatograph after UV broadening.

FIG. 12C provides Zimm plots for the first of two peaks as detected by MALS.

FIG. 12D provides Zimm plots for the second of two peaks as detected by MALS.

FIG. 12E provides Random Coil plots for the first of two peaks as detected by MALS.

FIG. 12F provides Random Coil plots for the second of two peaks as detected by MALS

FIG. 13 provides a chromatograph of a DNA digest at concentrations ranging from 0.1 μg to 5 μg separated according to some embodiments of the technology and detected using MALS.

FIG. 14 provides a representative chromatograph of a DNA digest separated using methods according to the present technology and detected using multi-angle light scattering (MALS) and dynamic light scattering (DLS) detectors.

FIG. 15 provides representative chromatographs for the separation of DNA from additional mixture components using a particle having a 125 Å pore size (top) or a 250 Å pore size (bottom).

FIG. 16 provides a chromatograph showing the theoretical separation of dsRNA and ssRNA present in a mixture relative to standard nucleic acid ladders.

FIG. 17 provides an exemplary graph depicting the resolution of nucleic acid species separations according to embodiments of the technology.

FIG. 18A, FIG. 18B, FIG. 18C, and FIG. 18D provide exemplary data depicting the resolution of nucleic acid species using 3.0 μm non-porous silica particles at 0.05 mL/min (FIG. 18A), 0.1 mL/min (FIG. 18B), 0.2 mL/min (FIG. 18C), or 0.3 mL/min (FIG. 18D).

FIGS. 19A-19D provide exemplary data depicting the resolution of nucleic acid species using 2.6 μm non-porous silica particles.

FIG. 19A uses a λ-DNA-HindIII digest with no additive in the mobile phase.

FIG. 19B uses a λ-DNA-HindIII digest with. 0.5% by volume 2-[2-(diethylamino)ethoxy]ethanol as a mobile phase additive.

FIG. 19C uses a λ-DNA Monocut mix with no additive in the mobile phase.

FIG. 19D uses a λ-DNA Monocut mix with 0.5% by volume 2-[2-(diethylamino)ethoxy]ethanol as a mobile phase additive.

FIG. 20A, FIG. 20B, and FIG. 20C provide exemplary data depicting the resolution of nucleic acid species separations using non-porous silica particles at acidic (pH=6; FIG. 20A), neutral (pH=7; FIG. 20B), and basic (pH=8; FIG. 20C) pH.

FIGS. 21A-21C provide an exemplary graph depicting the resolution of nucleic acid species separations using non-porous polymer particles at varying ionic strength.

FIG. 21A shows the chromatogram at 1× concentration of buffer (1× PBS).

FIG. 21B shows the chromatogram at 2× concentration of buffer (2× PBS).

FIG. 21C shows the chromatogram at 4× concentration of buffer (4× PBS).

FIG. 22 provides an exemplary graph depicting the resolution of nucleic acid species separations using superficially porous 3.0 μm hybrid solid core particles (FIG. 22).

FIG. 23 provides an exemplary graph depicting the resolution of nucleic acid species separations using 2.7 μm solid core silica particles.

FIG. 24A and FIG. 24B provide exemplary graphs depicting the resolution of nucleic acid species separations using 1.6 μm solid silica core particles bonded with T3 bonding (FIG. 24A-24B). FIG. 24A shows the results using a 1 kilobase pair DNA ladder. FIG. 24B shows the results using a λ-DNA BstEII digest.

FIG. 25A and FIG. 25B provide exemplary graphs depicting the resolution of nucleic acid species separations using 1.6 μm solid core silica particles (having a pore size of 90 Å). FIG. 25A shows the results using a 1 kilobase pair DNA ladder. FIG. 25B shows the results using a λ-DNA BstEII digest.

FIG. 26A and FIG. 26B provide exemplary graphs depicting the resolution of nucleic acid species separations using 1.6 μm solid silica particles (having a pore size of 120 Å). FIG. 26A shows the results using a 1 kilobase pair DNA ladder. FIG. 26B shows the results using a λ-DNA BstEII digest.

FIG. 27 provides an exemplary graph depicting the resolution of nucleic acid species separations using a diol functionalized particle surface at varying flow rates.

FIG. 28A provides an exemplary graph depicting the resolution of nucleic acid species separations using a sulfate functionalized surface. The oligonucleotides resolved in the gel-electrophoresis experiment in the inset are matched with the corresponding peak.

FIG. 28B, FIG. 28C, and FIG. 28D demonstrate the flow rate and ionic strength dependence of the chromatogram peaks observed in FIG. 28A.

FIG. 29A, FIG. 29B, FIG. 29C, and FIG. 29D provide an exemplary graph depicting the resolution of nucleic acid species separations using an amide functionalized surface.

FIG. 29E shows the oligonucleotides resolved in the gel-electrophoresis experiment.

FIG. 30A and FIG. 30B provide exemplary graph depicting the resolution of nucleic acid species separations using hydrophobic surface modifications. FIG. 30A shows a chromatogram obtained using an unfunctionalized surface. FIG. 30B shows a chromatogram obtained using a C1 XT functionalized surface.

### DETAILED DESCRIPTION

Disclosed herein are methods for the separation of nucleic acids under high-flow conditions (i.e., slalom chromatography). In order that the technology may be more readily understood, certain terms are first defined. It should be noted that whenever a value or range of values of a parameter are recited, it is intended that values and ranges intermediate to the recited values are part of this disclosure. The word “about” if not otherwise defined means ±5%. It is also to be noted that as used herein and in the claims, singular forms of “a”, “an”, and “the” include plural references unless the context clearly dictates otherwise. Additional terms are defined throughout the specification.

### Definitions

As used herein, the term “slalom chromatography” refers to a chromatographic method performed under high-flow conditions that separates a molecule by size based on hydrodynamic phenomena. The principles of slalom chromatography are further described in Hirabayashi and Kasai, J. Chromatogr. A (1996) 722(1-2):135-42 and Hirabayashi et al., Biochemistry (1990) 29(41):9515-21.

The high-flow rates used in slalom chromatography (SC) afford sufficiently high shear rates that result in prolonged extension of the molecules, e.g., nucleic acids, which is important for the separation of the molecules by size. Said extension can be assessed using the Weissenberg number. The Weissenberg number is a dimensionless metric that is a function of relaxation time and shear rate:

Wi=τ_{R}\dot{γ}
wherein τ_{R} is the relaxation time (seconds) as represented by the formula:

$${τ}_{R} = \frac{{nL}_{c}^{2} {I}_{P}}{{k}_{B} T}$$

wherein h is dynamic velocity (Pa*s); L_{C} is contour length (m); I_{P} is persistence length (m); K_{B} is the Boltzmann constant (1.38×10⁻²³ J.K⁻¹), and T is temperature (Kelvin); and
\dot{γ} is shear rate (seconds⁻¹) as represented by the formula:

$$\dot{γ} = \frac{Δ u}{Δ z} = \frac{16}{3} 〈 u 〉 / d$$

wherein Δu is the velocity difference between two points in a direction perpendicular to the flow direction, the two points separated by a distance of Δz;
   <u>is the average linear interstitial velocity along the packed bed; and
   d is the average flow-through tube diameter.

The relaxation time τ_{R} is dependent, in part, on intrinsic properties of the target nucleic acid. In certain instances, the relaxation time of a molecule under certain conditions is known in the art. For example, the L_{C} of linear dsDNA is equal to the number of base pairs * 3.4 Å; the dynamic velocity of linear dsDNA is 1.2 cP; and the persistence length of linear dsDNA is 450 Å. Alternatively, relaxation time of a molecule under certain conditions may be determined empirically using known methods that are readily understood by one of ordinary skill in the art. (see e.g., Bouchiat et al., Biophysical Journal (1999) 76:409-413).

As used herein, the term “nucleic acids” or “nucleic acid molecules” refers to a polymeric molecule comprising two or more nucleotides. Nucleic acids may comprise deoxyribonucleic acids (DNA), ribonucleic acids (RNA), or a combination thereof. DNA comprises the nucleotides cytidine, guanosine, adenosine, and thymidine. RNA comprises the nucleotides cytidine, guanosine, adenosine, and uridine. In some embodiments, the nucleic acid molecules may comprise nucleic acid analogues (i.e., non-naturally occurring nucleic acids or analogues thereof). Examples of nucleic acid analogues include peptide nucleic acids, locked nucleic acids, glycol nucleic acids, threose nucleic acids, hexitol nucleic acids. Nucleic acid analogues are further reviewed in Wang et al., Molecules (2023) 28(20):7043. Nucleic acid molecules may further be modified at the nucleobase, sugar, or phosphodiester backbone with an array of chemical modifications which are further reviewed in Epple et al., Emerg. Top. Life. Sci. (2021) 5(5):691-697.

Nucleic acid molecules range in length as measured by the number of nucleotides or base pairs. In embodiments of the present technology, the nucleic acids may range from about 5000 base pairs to about 100,000 base pairs. Nucleic acids may be single stranded (e.g., single-stranded DNA (ssDNA) or single-stranded RNA (ssRNA)). In some embodiments, the ssRNA is mRNA. Nucleic acids may be double stranded (e.g., double-stranded DNA (dsDNA) or double-stranded RNA (dsRNA). Double-stranded nucleic acids are made of complementary sequences (e.g., base-paired sequences) as is known in the art and would be readily understood by one of ordinary skill.

Nucleic acids may be present in different topologies, including linear and circular conformations. In some embodiments, circular nucleic acids may be supercoiled, in which the circular nucleic acid molecule undergoes additional twist strain.

As used herein, the term “porous” refers to a material that has a pore volume that is greater than 0.1 cc/g. Preferably, porous polymers have a pore volume that is greater than 0.1 cc/g (e.g., 0.5 cc/g). As used herein, the term “non-porous” refers to a material that has a pore volume that is less than 0.1 cc/g. Preferably, non-porous polymers have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA). In some embodiments, the pore volume is between 0.5 to 1.3 cc/g.

As used herein, the term “superficially porous particles” refers to a material that has a solid or nonporous core and an outer layer surrounding the core that is porous or has a greater degree of porosity that the nonporous core. In some embodiments, a porous or superficially porous particle described herein includes a pore volume of from about 20 Å to about 900 Å (e.g., about 45 Å).

As used herein, the term “zone retention factor,” also referred to as k₁, refers to the ratio of retention of an analyte (e.g., a nucleic acid molecule) on a column and the void retention volume. k₁ may be defined using the following formula:

$${k}_{1} = {k}_{1 , τ = 0} + ( 1 - {e}^{- τ / {τ}_{C}} ) ( {k}_{1 , MAX} - {k}_{1 , τ = 0} )$$

wherein k_{1,τ=0} refers to the retention of a molecule, e.g., a nucleic acid, in the absence of elongation (e.g., under low flow rates);
wherein T_{C} refers to the characteristic pressure stress of a molecule, e.g., a nucleic acid; and
wherein k_{1,MAX} refers to the maximum stretching of a molecule, e.g., a nucleic acid.

And the shear stress pressure variable is t=<u>h/dₚ. The above metrics may be determined empirically and can be a variable of flow rate, viscosity (e.g., viscosity of the mobile phase or temperature), and particle size.

As used herein, the term “efficiency” or “N” with respect to chromatography analysis refers to a measurement of peak dispersion. N may be defined using the following formula:

$$N = L / {d}_{p} ( \frac{1}{3} - \frac{3}{4} k + \frac{2}{3} {k}^{2} - \frac{1}{4} {k}^{3} )$$

wherein L is the column length;
wherein dₚ is the particle diameter; and
wherein k is the zone retention factor.

As used herein, the term “distinct peak” refers to a peak in a chromatogram, e.g., a detected analyte, that is baseline resolved.

As used herein, the term “selectivity factor” or “α” refers to the ability of a chromatographic method or system to distinguish analytes within a sample, typically a ratio of two peaks. α may be defined using the following formula:

$$α = ( {t}_{R 2} - {t}_{0} ) / ( {t}_{R 1} - {t}_{0} )$$

wherein t⁰ refers to the void elution time;
wherein t_{R1} and t_{R2} refer to the elution time of a first and second peak, respectively. In some embodiments, the selectivity factor is a comparison between two nucleic acid species.

A plot of the zone retention factor k₁=(t_{R}−tᵢ)/tᵢ, wherein t_{R} is as defined above and tᵢ is the interparticle hold-up time, as a function of τ is used to empirically determine the k₁ value.

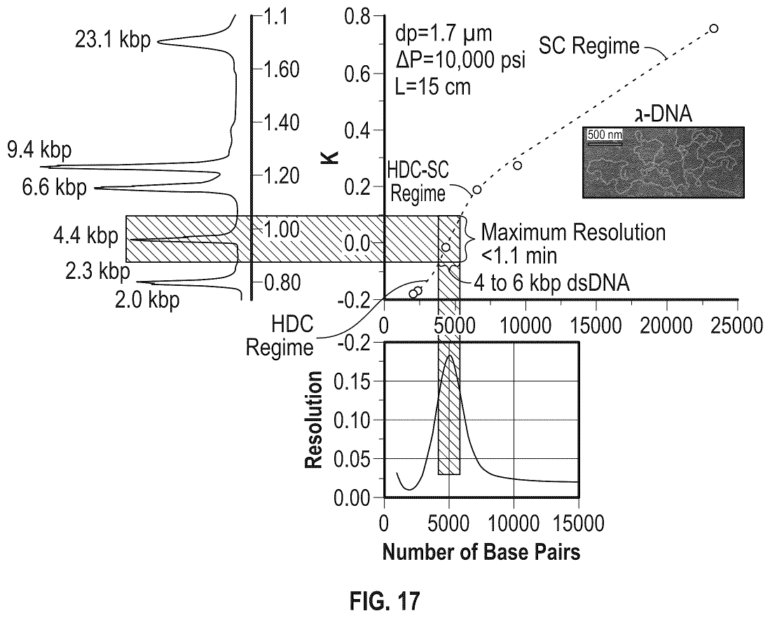

The separation of nucleic acids using the described methods can be achieved with high resolution by manipulating the particle size, column length, and column pressure. For example, but not by way of limitation, a particle size of 1.7 μm, column length of 10,000 psi, and column length of 15 cm affords maximum resolution in separating DNA between 4-6 kb in under 1.1 minutes (see, for example, FIG. 17). By adjusting said variables according to the teachings described herein and as shown in the examples (see, e.g., Example 4), maximum resolution can be achieved for DNA varying in nucleotide length.

### Methods and Conditions for Separating Nucleic Acids

In one aspect, disclosed herein are methods of separating nucleic acid species by size using slalom chromatography. Typically, nucleic acids are separated based on electrophoretic mobility using gel electrophoresis or analogous methods. These approaches can be time intensive (ranging from multiple hours to days) and are not sensitive enough to detect nucleic acid heterogeneity. These methods further hinder downstream processing of samples due to the need to extract nucleic acids from the gel itself (substantially lowering yield and increased contaminants). The present technology utilizes slalom chromatography which allows for rapid separation of nucleic acid species by size. As these methods utilize high performance liquid chromatography (HPLC) or ultra-high performance liquid chromatography (UHPLC) systems, the methods afford the separation and characterization of complex mixtures of nucleic acid species. The disclosed methods are capable of separating nucleic acids by size in under 10 minutes with high efficiency.

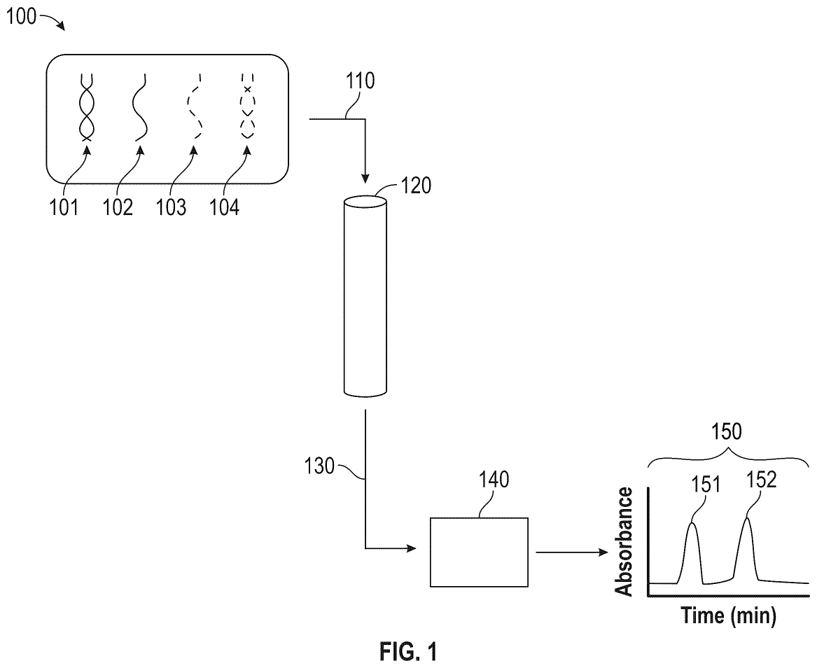

FIG. 1 illustrates an embodiment of the present disclosure. Sample 100, which may comprise double-stranded DNA (dsDNA; 101), single-stranded DNA (ssDNA; 102), single-stranded RNA (ssRNA; 103), double-stranded RNA (dsRNA; 104), or any combination thereof, are loaded (110) onto a column (120). Column 120 comprises a plurality of porous or non-porous particles. The sample is flowed through the column using a mobile phase and eluted (130). In preferred embodiments, the time between loading (110) and elution (130) is between 1-10 minutes. The sample is detected using a detector (140), for example an ultraviolet detector and a resultant chromatograph (150) is produced. The peaks in the chromatograph, for example peak 151 and peak 152 correspond to nucleic acid species of different sizes.

The methods disclosed herein utilize conditions that result in a high shear rate such that the nucleic acid molecules in the column are extended/elongated under the shear flow. Extension or elongation of nucleic acid molecules can be determined by the Weissenberg number, which compares elastic and viscous forces exerted on a molecule. In instances wherein the Weissenberg number is greater than 1, the nucleic acid molecule is extensible under the shear flow and would be separated by size based on the principles of slalom chromatography. In instances wherein the Weissenberg number is less than 1, the nucleic acid molecule is not extensible under the shear flow and would not be separated by size based on the principles of slalom chromatography.

Accordingly, in one aspect the methods disclosed herein utilize conditions that result in the nucleic acid molecules exhibiting a Weissenberg number (Wi) of greater than 1. In some embodiments, the Wi is between 1-100, 5-100, 10-100, 15-100, 20-100, 25-100, 30-100, 35-100, 40-100, 45-100, 55-100, 60-100, 65-100, 70-100 75-100, 80-100, 85-100, 90-100, or 95-100.

Shear rate, and consequently the Weissenberg number (Wi), can be influenced by several parameters, including flow rate, particle size, mobile phase viscosity, and temperature. Thus, said variables may be adjusted to achieve sufficient Wi numbers for the separation of the desired nucleic acid molecules.

In some embodiments, the flow rate is between 0.1 mL/min and 15 mL/min. In some embodiments, the flow rate is 0.1-0.5 mL/min, 0.5-1.0 mL/min, 1.0-1.5 mL/min, 1.5-2.0 mL/min, 2.0-2.5 mL/min, 2.5-3.0 mL/min, 3.0-3.5 mL/min, 3.5-4.0 mL/min, 4.0-4.5 mL/min, 4.5-5.0 mL/min, 5.0-5.5 mL/min, 5.5-6.0 mL/min, 6.0-6.5 mL/min, 6.5-7.0 mL/min, 7.0-7.5 mL/min, 7.5-8.0 mL/min, 8.0-8.5 mL/min, 8.5-9.0 mL/min, 9.0-9.5 mL/min, 9.5-10 mL/min, 10-10.5 mL/min, 10.5-11 min/min, 11.5-12 mL/min, 12-12.5 mL/min, 12.5-13 mL/min, 13-13.5 mL/min, 13.5-14 mL/min, 14-14.5 mL/min, or 14.5-15 mL/min. In general, increased flow rates increases shear rates.

In some embodiments, the plurality of particles comprise a diameter of between about 1 μm and about 10.5 μm. In some embodiments, the plurality of particles comprise a diameter of between about 1-2 μm, 2-3 μm, 3-4 μm, 4-5 μm, 5-6 μm, 6-7 μm, 7-8 μm, 8-9 μm, 9-10 μm, or 10-10.5 μm. In some embodiments, the plurality of particles comprise a diameter of between about 1.5-1.9 μm. In some embodiments, the plurality of particles comprise a diameter of between about 2.8-3.2 μm. In some embodiments, the plurality of particles comprise a diameter of between about 4.8-5.2 μm. In some embodiments, the plurality of particles comprise a diameter of between about 9.8-10.2 μm. In some embodiments, the plurality of particles comprise a diameter of about 1.7 μm. In some embodiments, the plurality of particles comprise a diameter of about 3.0 μm. In some embodiments, the plurality of particles comprise a diameter of about 5.0 μm. In some embodiments, the plurality of particles comprise a diameter of about 10 μm. In general, larger particle sizes are used to separate larger nucleic acid molecules, and smaller particle sizes are used to separate smaller nucleic acid molecules. Example 4 and Tables 2-5 demonstrate the effects of particle size and flow rate on the ability to separate different sized nucleic acid molecules.

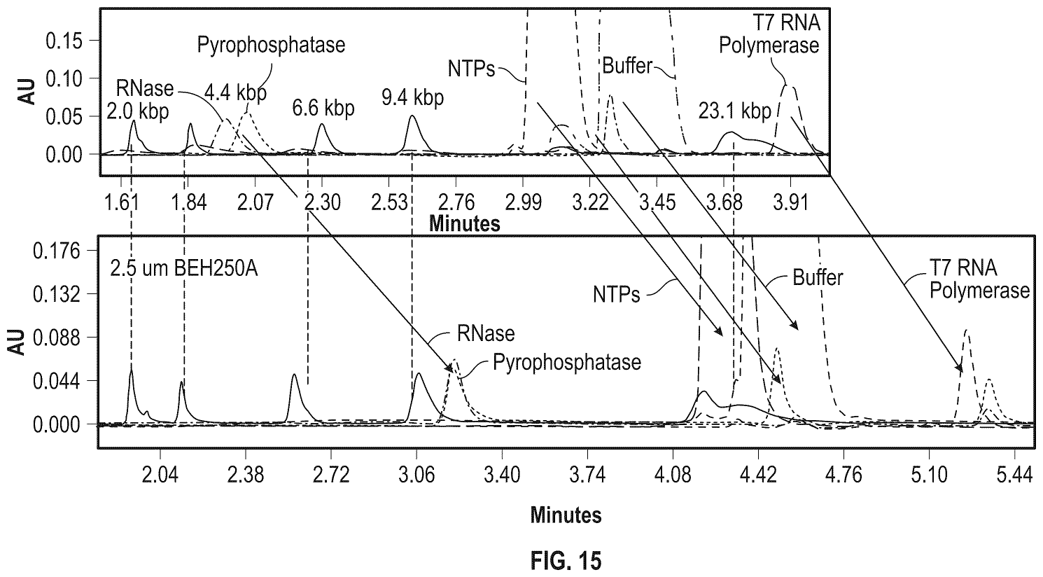

In some embodiments, the particles have an average pore diameter of between 100 Å to 900 Å. Without wishing to be bound by any particular theory, increasing the pore size shifts the retention of larger molecules relative to the smaller nucleic acid species. Example 9 and FIG. 15 demonstrate the effect of pore size on the retention of nucleic acid species and additional larger components, such as proteins and buffer components.

In some embodiments, the viscosity of the mobile phase is adjusted. Methods of altering the viscosity of the mobile phase are known in the art, and include, for example, the inclusion of a sugar (e.g., sucrose). In general, increased viscosity increases shear rates. A number of mobile phase buffers are suitable for use with the disclosed methods as would be understood by one of ordinary skill in the art. In some embodiments, the buffer is phosphate-buffered saline. In some embodiments, the phosphate-buffered saline is at 1× concentration, at 2× concentration, at 0.1× concentration, at 0.01× concentration, or any value between 0.01× and 2× concentration.

In some embodiments, the temperature at which the separation is performed is altered. In some embodiments, the temperature is between 25° C. and 50° C. In some embodiments, the temperature is between 25-30° C., 30-35° C., 35-40° C., 40-45° C., and 45-50° C. In general, increases in temperature reduce shear rates.

### Methods of Separating DNA, RNA, or Mixtures Thereof

The methods disclosed herein may be used for the separation of nucleic acids, including DNA, RNA, or mixtures thereof. The nucleic acids may be ssDNA, dsDNA, ssRNA, dsRNA, and may occur in any known topology, including linear, circular, or supercoiled. The nucleic acids may be between 1,000-100,000 base pairs in length. In some embodiments, the nucleic acids are between 1000-2000, 2000-3000, 3000-4000, 4000-5000, 5000-7500, 7500-10,000, 10,000-12,500, 12,500-15,000, 15,000-17,500, 17,500-20,000, 20,000-25,000, 25,000-30,000, 30,000-35,000, 35,000-40,000, 40,000-45,000, 45,000-50,000, 50,000-55,000, 55,000-60,000, 60,000-65,000, 65,000-70,000, 70,000-75,000, 75,000-80,000, 80,000-85,000, 85,000-90,000, 90,000-95,000, or 95,000-100,000.

Accordingly, in one aspect disclosed herein is a method of separating a sample comprising DNA and RNA. The method comprises loading a sample comprising DNA and RNA onto a chromatographic column comprising a plurality of porous or non-porous particles, eluting the DNA and RNA from the column, and detecting the DNA and RNA in the eluent. In some embodiments, the DNA is dsDNA and/or ssDNA. In some embodiments, the RNA is dsRNA or ssRNA. In some embodiments, the DNA and RNA are eluted from the column in between about 1-10 minutes, more preferably 1-3 minutes. In some embodiments, the DNA and RNA are eluted with a zone retention factor of less than 0.3. In some embodiments, the eluting is performed with an efficiency of greater than 40,000. Example 4 describes methods of separating a sample comprising DNA and RNA according to some embodiments of the technology.

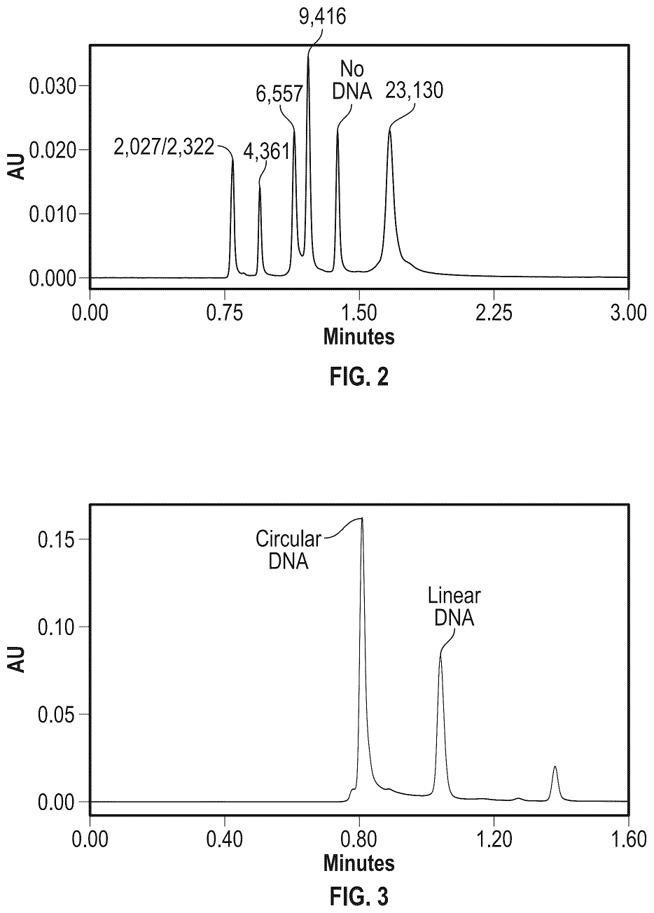

In another aspect, disclosed herein is a method of separating a sample comprising DNA. The method comprises loading a sample comprising DNA onto a chromatographic column comprising a plurality of porous or non-porous particles, eluting the DNA from the column, and detecting the DNA in the eluent. In some embodiments, the DNA is dsDNA and/or ssDNA. In some embodiments, the DNA is eluted from the column in between about 1-10 minutes, more preferably 1-3 minutes. In some embodiments, the DNA is eluted with a zone retention factor of less than 0.3. In some embodiments, the eluting is performed with an efficiency of greater than 40,000. Examples 1-2 and FIGS. 2-3 describe a method of separating a sample comprising DNA according to some embodiments of the present technology.

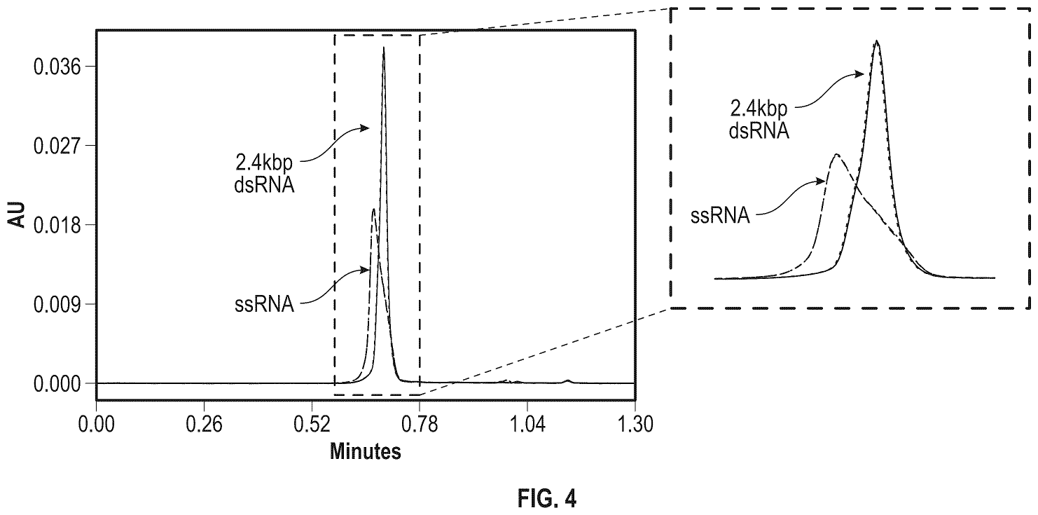

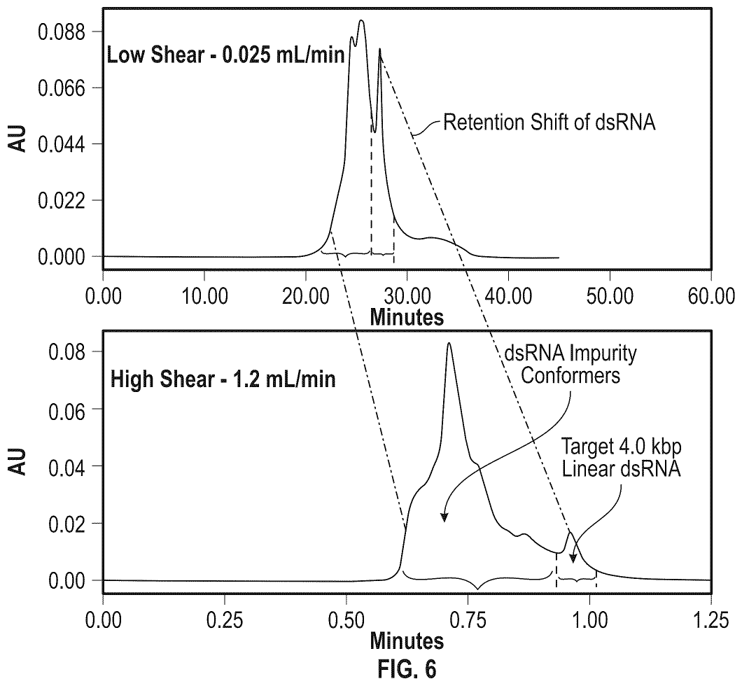

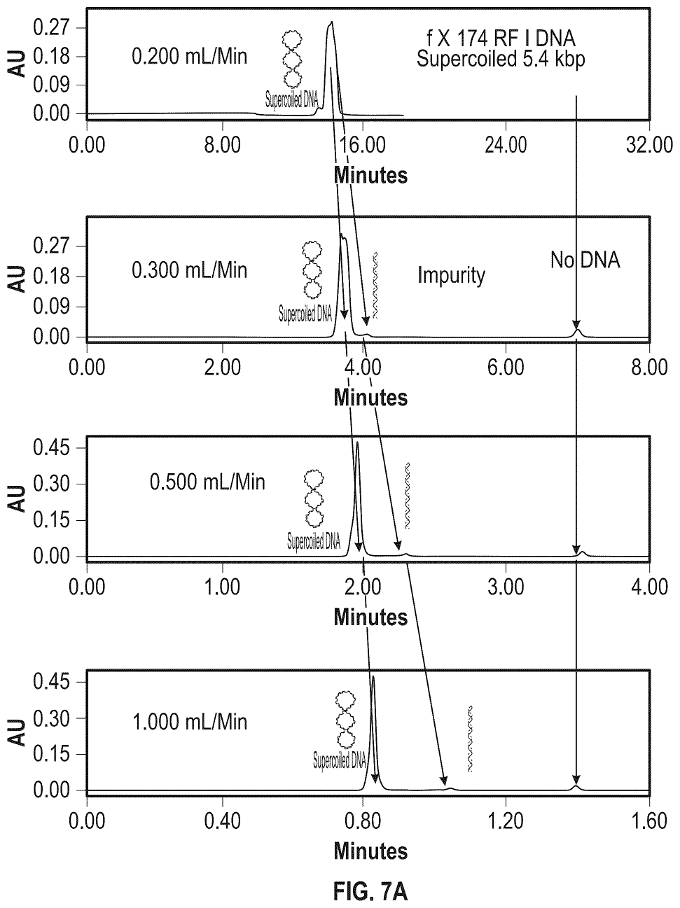

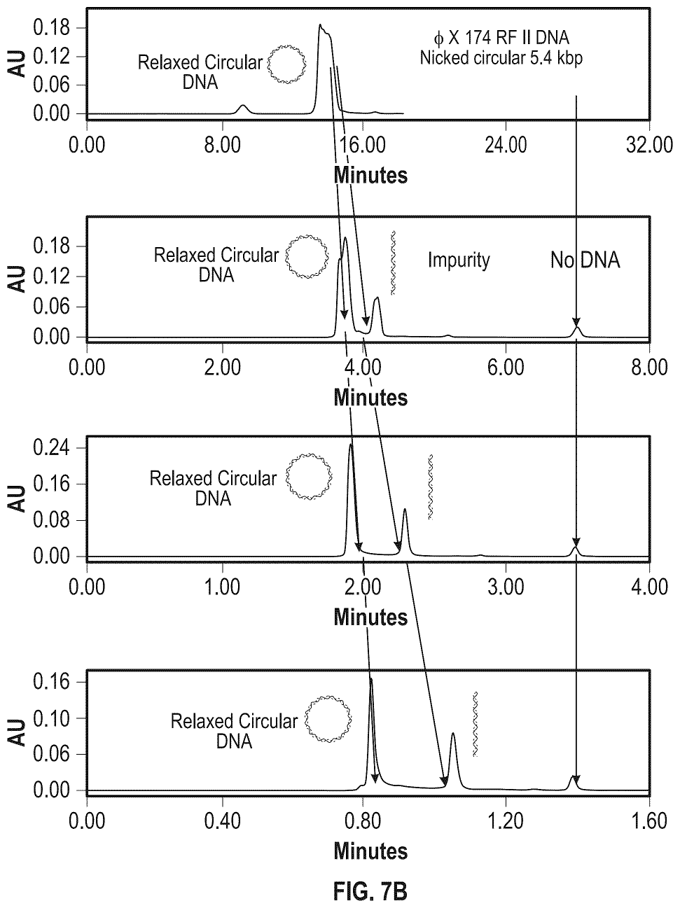

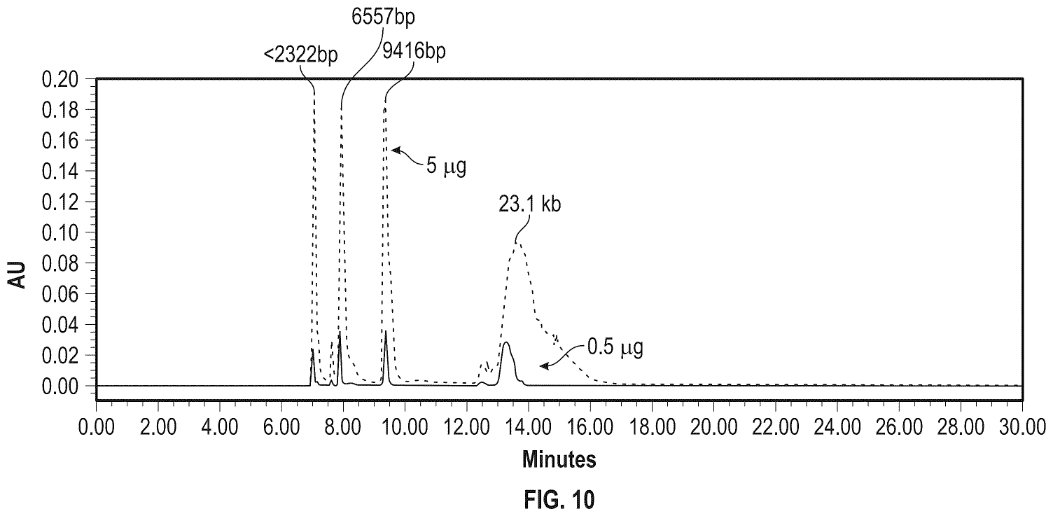

In another aspect, disclosed herein is a method of separating a sample comprising RNA. The method comprises loading a sample comprising RNA onto a chromatographic column comprising a plurality of porous or non-porous particles, eluting the RNA from the column, and detecting the RNA in the eluent. In some embodiments, the RNA is dsRNA and/or ssRNA. In some embodiments, the DNA is eluted from the column in between about 1-10 minutes, more preferably 1-3 minutes. In some embodiments, the DNA is eluted with a zone retention factor of less than 0.3. In some embodiments, the eluting is performed with an efficiency of greater than 40,000. Example 3 and FIG. 4 describe a method of separating a sample comprising RNA according to some embodiments of the present technology. Example 6 and FIG. 6 describe a method of separating linear dsRNA from other dsRNA impurity conformers present in a sample. Example 7 and FIG. 7A-7B describe a method of separating supercoiled DNA or circular DNA from linear DNA impurities. Example 8 and FIGS. 10-11, 12A-12D, and 13-14 describe a method of separating DNA fragments and detecting said fragments with multi-angle light scattering (MALS) and/or dynamic light scattering (DLS).

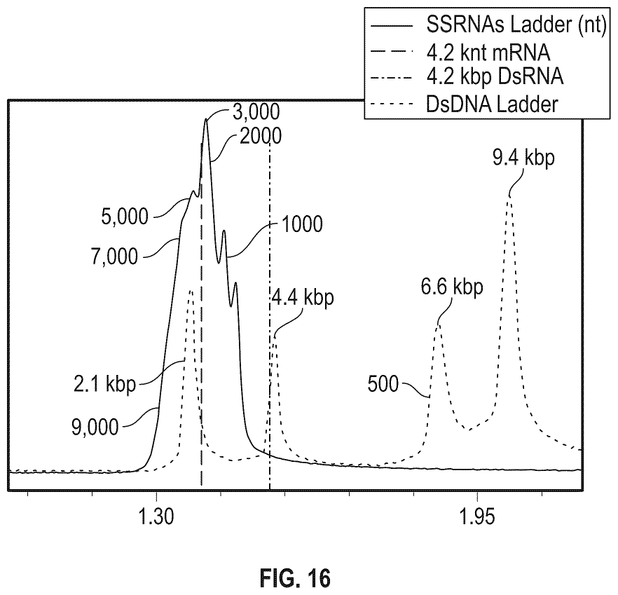

For example, but not by way of limitation, the methods described herein may be used to separate double-stranded RNA (dsRNA) from single-stranded RNA (ssRNA) present in a mixture. As shown in FIG. 16, a sample comprising a 4.2 kb dsRNA and 4.2 kb ssRNA would be resolved using the methods described herein.

In some embodiments of the above methods, the eluent is collected for downstream processing. Examples of downstream processing include, but are not limited to, sequencing of the nucleic acid molecules or cloning of the nucleic acid molecules using methods known in the art.

### Chromatography Systems

The methods disclosed herein may be used with any high-performance liquid chromatography (HPLC) or ultra-high performance liquid chromatography (UHPLC) system. Said systems comprise a column comprising a plurality of particles. The plurality of particles may be porous or non-porous. In some embodiments, the porous or non-porous particles are inorganic silica particles, organic particles, or inorganic/organic hybrid particles. Examples of particles suitable for use include, but are not limited to, ethylene bridged hybrid particles (BEH; comprising tetraethoxysilane (TEOS) and bis(triethoxysilyl) ethane as described in U.S. Pat. Nos. 6,686,035, and 7,250,214, incorporated herein by reference. Additional particle compositions include, for example, hybrid inorganic/organic particles such as those described in U.S. Pat. Nos. 11,291,974 and 9,145,481. Additional particles may include polystyrene, divinylbenzene, polyacrylamides, and polymethylacrylates with varying degrees of crosslinking.

As described above, the plurality of particles may comprise a diameter of between about 1 μm and about 10.5 μm. In some embodiments, the plurality of particles comprise a diameter of between about 1-2 μm, 2-3 μm, 3-4 μm, 4-5 μm, 5-6 μm, 6-7 μm, 7-8 μm, 8-9 μm, 9-10 μm, or 10-10.5 μm. In some embodiments, the plurality of particles comprise a diameter of between about 1.5-1.9 μm. In some embodiments, the plurality of particles comprise a diameter of between about 2.8-3.2 μm. In some embodiments, the plurality of particles comprise a diameter of between about 4.8-5.2 μm. In some embodiments, the plurality of particles comprise a diameter of between about 9.8-10.2 μm. In some embodiments, the plurality of particles comprise a diameter of about 1.7 μm. In some embodiments, the plurality of particles comprise a diameter of about 3.0 μm. In some embodiments, the plurality of particles comprise a diameter of about 5.0 μm. In some embodiments, the plurality of particles comprise a diameter of about 10 μm.

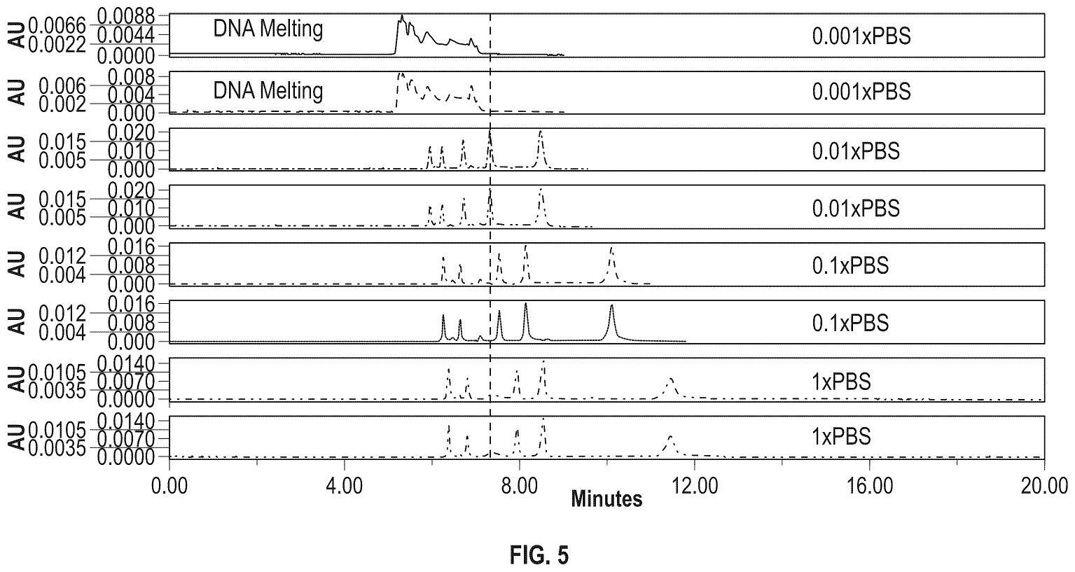

In some embodiments, the particle comprises a negatively charged surface, which contributes to electrostatic repulsion of the negatively charged nucleic acid molecules and the particle surface. Without wishing to be bound by any particular theory, distance of the nucleic acid molecules from the particle surface sharpens peak widths and increases separation speed at a constant resolution. Said electrostatic repulsion can further be adjusted by altering the ionic strength of the mobile phase, e.g., by increasing or decreasing the salt content of the mobile phase. Example 5 and FIG. 5 demonstrate the impact of ionic strength on elution time and peak resolution (i.e., changes to the k₁ value of said peaks) when separating nucleic acids, such as linear, double-stranded DNA. Examples of negatively charged surfaces include silanols, surface modifications with a silane comprising a pendant group that has a negative charge (e.g., COO⁻ or SO₃⁻), the grafting of a negatively charged group onto a polymer particle, or a chemical modification of a polymer particle that results in the surface display of a negatively charged group as would be understood by one of ordinary skill in the art.

In some embodiments, the columns feature non-porous particles. Other embodiments feature porous particles. In these embodiments, analyte size becomes relevant to k₁ considerations. For instance, in embodiments in which analyte size (e.g., length, width, volume, etc.) is larger than the size of individual pores within the particles, the k₁ value can be calculated and evaluated as described above. However, if the analyte size is less than the pore size (e.g., DNA size is less than the size of the porosity (e.g., 3000 Å), then a correction is needed to account for the possible penetration of the analyte inside the particle volume. In that case involving particle porosity sized larger than the analyte, there would be a mixed retention mechanism shifting the value of the zone retention factor as calculated by the “universal retention factor formula”, defined herein and throughout as:

$${k}_{1} = ( 1 - ∅ ) {k}_{1 , H D C} + ∅ {k}_{1 , SC} + {k}_{1 , SEC}$$

In the above equation, k_{1,SEC}, e.g., retention wherein SEC principles are at play, may be defined by the following formula:

$${k}_{1 , SEC} = \frac{1 - {ε}_{e}}{{ε}_{e}} {ε}_{p} ( 1 - 4.4 ( \frac{{R}_{g}}{{D}_{m e s o}} ) + 4.98 {( \frac{{R}_{g}}{{D}_{m e s o}} )}^{2} )$$

wherein R_{g} is the gyration radius of the DNA as defined below,
Dₘₑₛₒ refers to the size of the mesopores on the particle, and wherein εₑ is the inter-particle void fraction of the column, and εₚ is the internal porosity of the particle.

In the above equation, k_{1,HDC}, e.g., retention wherein HDC principles are at play, may be defined by the following formula:

$${k}_{1 , H D C} = \frac{1}{1 + 2 ( \frac{{D}_{r e p}}{{D}_{p o r e}} ) - 2.698 ( {( \frac{{D}_{r e p}}{{D}_{p o r e}} )}^{2} )} - 1$$

wherein Dₚₒᵣₑ refers to the average distance between particles, and wherein Dᵣₑₚ is defined by the following formula:

$${D}_{r e p} = 4 \sqrt{\frac{{( \frac{\sqrt{π}}{2} {R}_{g} )}^{3}}{3 〈 {L}_{e x t} 〉}}$$

and wherein R_{g} is the gyration radius as defined by:

$$R g = 0.116 * L {c}^{0.57}$$

and <Lₑₓₜ>is the average extension length of the DNA under shjear flow conditions.

In the above equation, k_{1,SC}, e.g., retention wherein SC principles are at play, may be defined by the following formula:

$${k}_{1 , SC} = ( 1 - {e}^{- \frac{τ}{{τ}_{C}}} ) * α * {L}_{c o n t o u r}$$

wherein α is a proportionality empirical constant independent of the contour length of the DNA (α=0.1 mm⁻¹), τ_{C} is a characteristic shear stress of the DNA chain (τ_{C}=0.25 Pa for dsDNA) and the remaining variables are as defined above. In the context of the Universal Retention Factor Formula, k_{1,SC} defines the variable <Lₑₓₜ>.

Without wishing to be bound by any particular theory, it is understood that multiple chromatographic principles may influence retention of an analyte (e.g., a DNA or RNA molecule) based on the size and relaxation time (ms) of the analyte. For example, under increased flow rate (i.e., high shear rates), analytes with longer relaxation times will have increased retention on a column. In this context, k₁ may be defined using the universal retention factor formula described above.

The column material may be stainless steel, polyetheretherketone (PEEK) lined steel, titanium, or a stainless alloy. Column inner diameters may range from about 2.1 mm to about 7.8 mm. Column lengths may range from about 10 mm to about 300 mm. Exemplary column dimensions include, but are not limited to, 2.1×20 mm, 2.1×50 mm, 2.1×100 mm, 2.1×150 mm, 4.6×50 mm, 4.6×100 mm, 4.6×150 mm, and 4.6×300 mm.

The choice of column and particle, particularly with respect to column length and particle size, is in part dependent on the size of the nucleic acid molecules to be separated and detected as would be appreciated and readily understood by one of ordinary skill in the art.

According to embodiments of the present disclosure, the columns are connected in fluidic series to a detector. In one aspect, the detector is an ultraviolet (UV) or a tunable ultraviolet (TUV) detector. In some embodiments, the detector is a multi-angle light scattering (MALS) detector. In some embodiments, the UV or TUV detector measures at between 210 nm to 300 nm. In preferred embodiments, the UV or TUV detector measures at between 230 to 260 nm, or more preferably at 230 nm and 260 nm. Said wavelengths are known in the art to detect nucleic acid molecules, including DNA and RNA. Additional detectors, such as fluorescence spectroscopy or mass spectrometry detectors can be utilized in conjunction with the disclosed methods. The detectors can be used alone or in tandem and can be further adjusted to detect molecule(s) of interest. For example, and not by way of limitation, a fluorescence detector may be utilized if the sample comprises a fluorescent molecule of interest.

In some embodiments, an interior surface of the column is treated to reduce non-specific binding and enhance overall efficiency of the chromatographic system. In particular, an alkylsilyl coating or other high performance surface is provided to limit or reduce non-specific binding of a sample with walls or interior surfaces of a column body. Without wishing to be bound by theory, it is believed that an alkylsilyl coating covering metal surfaces prevents or minimizes contact between fluids passing through the column body and the interior surfaces of the column. Typically, the alkylsilyl coating is applied to metal surfaces defining what is known as a wetted path of the column. A metal wetted path includes all surfaces formed from metal that are exposed to fluids during operation of the chromatographic column. The metal wetted path includes not only column body walls but also metal frits disposed within the column.

In general, the alkylsilyl coating is applied through a vapor deposition technique. Precursors are charged into a reactor in which the part to be coated is located. Vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating comprises a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. In some embodiments, the high-performance surface is a C2-PEG. A C2-PEG coating may be prepared as described in U.S. Publication No. 2022/0118443. For example, an organosilane precursor, such as bis(trichlorosilyl)ethane or bis(trimethoxysilyl)ethane, may first be vapor deposited on a metal surface, including an interior surface of the column or a metal frit of the column. Following vapor deposition of the organosilane precursor, the coated metal components may be treated with a toluene solution of a polyethylene glycol (PEG), for example 2-[methoxy(polyethyleneoxy)₆₋₉propyl]tris(dimethylaminosilane). The reagent solution may be reacted with the metal components for ˜3 days, washed in toluene, washed in isopropanol, and vacuum dried at 70° C., resulting in a C2-PEG coating. Other embodiments of alkylsilyl coatings suitable for use with the present technology are described in U.S. Patent Publication No. 2019/0086371 and U.S. Application Publication No. 2022/0118443 (which are hereby incorporated by reference).

### EXAMPLES

### Example 1: Separation of Linear Double-Stranded DNA

Linear double-stranded DNA was separated using the methods disclosed herein. A Lambda DNA (A-DNA) double-stranded DNA (dsDNA) sample (48,502 base pairs/nucleotides in length) was used (available from Thermo Scientific™). The sample was digested using a restriction enzyme to produce six, linear dsDNA fragments, of approximately 2,027, 2,322, 4,361, 6,557, 9,416, and 23,130 base pairs in length. The sample was loaded onto a 4.6 mm×15 cm column comprising porous 1.7 μm BEH particles having 45 Å pore sizes. The column hardware was coated with a C2-PEG high-performance surface. The samples were flowed through the column with a flow rate of 1 mL/min using 100 mM phosphate-buffered saline (pH 8). As shown in FIG. 2, the method afforded robust separation and baseline resolved peaks of the expected dsDNA fragments (with the 2,027 and 2,322 fragments co-eluting) in under 2.25 minutes. The peak labeled no DNA is due to small molecules present in the sample and entering the mesopores of the 1.7 μm BEH45 particles. The k₁ value for the 2,027 and 2,322 bp peak was −0.16; for the 4,361 bp peak was 0.01; for the 6,557 bp peak was 0.24; for the 9,416 bp peak was 0.31; and for the 23,130 bp peak was 0.8. Increasing the flow rate or particle size could afford a lower k1 value for the 23,130 bp fragment.

### Example 2: Separation of Circular and Linear Double-Stranded DNA

The ability to separate DNA molecules with different topologies using the methods disclosed herein was determined. A nicked, circular double-stranded DNA fX174 RF II, 5,386 sample (5,386 base pairs/nucleotides in length) was used (available from NEB). ˜90% of the sample comprises nicked circular DNA, and the remainder is supercoiled or linear.

The sample was loaded onto a 4.6 mm×15 cm column comprising porous 1.7 μm BEH particles having 45 Å pore sizes. The column hardware was coated with a C2-PEG high-performance surface. The samples were flowed through the column with a flow rate of 1 mL/min using 100 mM phosphate-buffered saline (pH 8). As shown in FIG. 3, the method afforded robust selectivity between the circular and linear forms of the DNA sample in under 1.2 minutes. In contrast, separating the same sample using size-exclusion chromatography resulted in poor selectivity between the two forms with long elution times of 12-15 minutes (data not shown). Thus, the methods disclosed herein are capable of separating similar length samples with differing topologies. The k₁ value for this separation was 0.15 for the linear DNA. The unlabeled, third peak did not comprise DNA.

### Example 3: Separation of Single- and Double-Stranded RNA

The ability to separate single-stranded RNA (ssRNA) and double-stranded RNA (dsRNA) using the methods disclosed herein was determined. A KH20 RNA sample, ˜2,400 base pairs/nucleotides in length, was used (available from New England Biolabs).

The sample was loaded onto a 4.6 mm×150 mm column comprising porous 1.7 μm BEH particles having 45 Å pore sizes. The column hardware was coated with a C2-PEG high-performance surface. The samples were flowed through the column with a flow rate of 1 mL/min using 100 mM phosphate-buffered saline (pH 8). As shown in FIG. 4, the method could distinguish between ssRNA and dsRNA forms of the RNA sample in under 1 minute (with the dashed box representing a blown-out portion of the chromatograph). As such, the disclosed methods afford rapid detection of ssRNA purity in a sample. The k₁ value for this separation was −0.24 for the 2.4 kb dsRNA sample.

### Example 4: Separation of DNA/RNA by Flow Rate and Particle Size

The relationship between particle size, flow rate, and nucleic acid size was determined. Single-stranded RNA (ssRNA), single-stranded DNA (ssDNA), double-stranded RNA (dsRNA), and double-stranded DNA (dsDNA) of varying lengths were tested, and selectivity factors for the separation of the double-stranded molecules from the single-stranded molecules (α_{ds/ss}) or the separation of the double-stranded DNA from the double-stranded RNA (α_{ds2/ds1}) were determined. The experiments were repeated using particle sizes of 1.7 μm, 3.0 μm, 5.0 μm, and 10 μm, with a range of flow rates using a 4.6 mm inner diameter column. The particles were BEH porous (average pore size of ˜125 Å). The column hardware was coated with a C2-PEG high-performance surface.

A summary of the relaxation time (T_{R}) and the gyration radius (R_{G}) for the DNA/RNA molecules tested is shown in Table 1.

TABLE 1
Summary of Parameters for Tested Nucleic Acids

| | | Nucleic Acid Length | | | | | |
|---|---|---|---|---|---|---|---|
| | | (kilonucleotides/kilobasepairs) | | | | | |
| Nucleic Acid | Parameter | 1 | 5 | 10 | 25 | 50 | 100 |
| ssDNA/ssRNA | Relaxation | 0.015 | 0.2 | 0.8 | 4 | 12 | 40 |
| | Time (ms) | | | | | | |
| | Gyration | 0.023 | 0.052 | 0.073 | 0.12 | 0.16 | 0.23 |
| | Radius (μm) | | | | | | |
| dsDNA/dsRNA | Relaxation | 0.4 | 6 | 20 | 90 | 300 | 1000 |
| | Time (ms) | | | | | | |
| | Gyration | 0.063 | 0.16 | 0.23 | 0.39 | 0.58 | 0.87 |
| | Radius (μm) | | | | | | |

The ability to separate the nucleic acid molecules were first tested using a 1.7 μm particles at flow rates 0.1 mL/min, 0.5 mL/min, or 1.0 mL/min. When relevant, selectivity factors were also calculated using hydrodynamic chromatographic principles (HDC; when the γ shear rate trends towards zero). Selectivity factors were determined as described above. As shown in Table 2, in general, a particle size of 1.7 μm and a flow rate of >0.5 mL/min afforded good separation as measured by selectivity factors for nucleic acid molecules between 5,000 bp/nt and 25,000 bp/nt, including both between double-stranded species and between single-and double-stranded species.

TABLE 2
Specificity Factors Using a 1.7 μm Particle

| | | | Nucleic Acid Length | | | | | |
|---|---|---|---|---|---|---|---|---|
| Flow | Selectivity | | (kilonucleotides/kilobasepairs) | | | | | |
| Rate | Factor | Method | 1 | 5 | 10 | 25 | 50 | 100 |
| 0.1 | α_{ds/ss} | HDC | 0.89 | — | — | — | — | — |
| mL/min | | SC | 1.00 | 1.01 | 1.05 | 1.27 | 1.68 | — |
| | α_{ds2/ds1} | HDC | — | — | — | — | — | — |
| | | SC | — | 1.00 | 1.01 | 1.05 | 1.14 | — |
| 0.5 | α_{ds/ss} | HDC | 0.89 | — | — | — | — | — |
| mL/min | | SC | 1.00 | 1.14 | 1.36 | 2.04 | — | — |
| | α_{ds2/ds1} | HDC | — | — | — | — | — | — |
| | | SC | — | 1.14 | 1.19 | 1.51 | — | — |
| 1.0 | α_{ds/ss} | HDC | 0.89 | — | — | — | — | — |
| mL/min | | SC | 1.02 | 1.21 | 1.47 | 2.24 | — | — |
| | α_{ds2/ds1} | HDC | — | — | — | — | — | — |
| | | SC | — | 1.19 | 1.22 | 1.56 | — | — |

The ability to separate the nucleic acid molecules was then tested using 3.0 μm particles at flow rates 0.25 mL/min, 0.75 mL/min, or 1.5 mL/min. When relevant, selectivity factors were also calculated using hydrodynamic chromatography (HDC; a method that differs from slalom chromatography due to the low shear rates). Selectivity factors were determined as described above. As shown in Table 3, in general, a particle size of 3 μm and a flow rate of >0.25 mL/min afforded good separation as measured by selectivity factors for nucleic acid molecules between 5,000 bp/nt and 50,000 bp/nt, including both between double-stranded species and between single-and double-stranded species. A flow rate of 0.25 mL/min was capable of separating nucleic acid species up to 100 kbp/knt in length.

TABLE 3
Specificity Factors Using a 3.0 μm Particle

| | | | Nucleic Acid Length | | | | | |
|---|---|---|---|---|---|---|---|---|
| Flow | Selectivity | | (kilonucleotides/kilobasepairs) | | | | | |
| Rate | Factor | Method | 1 | 5 | 10 | 25 | 50 | 100 |
| 0.25 | α_{ds/ss} | HDC | 0.92 | 0.86 | 0.86 | — | — | — |
| mL/min | | SC | 1.00 | 1.01 | 1.06 | 1.23 | 1.54 | 2.15 |
| | α_{ds2/ds1} | HDC | — | 0.88 | 0.96 | — | — | — |
| | | SC | — | 1.01 | 1.04 | 1.17 | 1.26 | 1.42 |
| 0.75 | α_{ds/ss} | HDC | 0.92 | 0.86 | 0.86 | — | — | — |
| mL/min | | SC | 1.00 | 1.07 | 1.19 | 1.54 | 2.12 | — |
| | α_{ds2/ds1} | HDC | — | 0.88 | 0.96 | — | — | — |
| | | SC | — | 1.07 | 1.11 | 1.31 | 1.41 | — |
| 1.5 | α_{ds/ss} | HDC | 0.92 | 0.86 | 0.86 | — | — | — |
| mL/min | | SC | 1.01 | 1.11 | 1.25 | 1.68 | 2.29 | — |
| | α_{ds2/ds1} | HDC | — | 0.88 | 0.96 | — | — | — |
| | | SC | — | 1.10 | 1.13 | 1.36 | 1.45 | — |

The ability to separate the nucleic acid molecules was then tested using 5.0 μm particles at flow rates 1.0 mL/min, 2.5 mL/min, or 5.0 mL/min. When relevant, selectivity factors were also calculated using hydrodynamic chromatography (HDC; a method that differs from slalom chromatography due to the low shear rates). Selectivity factors were determined as described above. As shown in Table 4, in general, a particle size of 5 μm and a flow rate of >1.0 mL/min afforded good separation as measured by selectivity factors for nucleic acid molecules between 5,000 bp/nt and 50,000 bp/nt, including both between double-stranded species and between single-and double-stranded species. A flow rate of 1.0 mL/min was capable of separating nucleic acid species up to 100 kbp/knt in length.

TABLE 4
Specificity Factors Using a 5.0 μm Particle

| | | | Nucleic Acid Length | | | | | |
|---|---|---|---|---|---|---|---|---|
| Flow | Selectivity | | (kilonucleotides/kilobasepairs) | | | | | |
| Rate | Factor | Method | 1 | 5 | 10 | 25 | 50 | 100 |
| 1.0 | α_{ds/ss} | HDC | 0.94 | 0.89 | 0.86 | 0.86 | — | — |
| mL/min | | SC | 1.0 | 1.03 | 1.09 | 1.29 | 1.60 | 2.12 |
| | α_{ds2/ds1} | HDC | — | 0.90 | 0.94 | 0.95 | — | — |
| | | SC | — | 1.03 | 1.06 | 1.18 | 1.27 | 1.43 |
| 2.5 | α_{ds/ss} | HDC | 0.94 | 0.89 | 0.86 | 0.86 | — | — |
| mL/min | | SC | 1.0 | 1.07 | 1.15 | 1.40 | 1.74 | — |
| | α_{ds2/ds1} | HDC | — | 0.9 | 0.94 | 0.95 | — | — |
| | | SC | — | 1.03 | 1.06 | 1.18 | 1.27 | — |
| 5.0 | α_{ds/ss} | HDC | 0.94 | 0.89 | 0.86 | 0.86 | — | — |
| mL/min | | SC | 1.01 | 1.08 | 1.17 | 1.40 | 1.69 | 2.12 |
| | α_{ds2/ds1} | HDC | — | 0.90 | 0.94 | 0.95 | — | — |
| | | SC | — | 1.07 | 1.09 | 1.25 | 1.34 | — |

The ability to separate the nucleic acid molecules was lastly tested using 10 μm particles at flow rates 2.5 mL/min, 7.5 mL/min, or 15 mL/min. When relevant, selectivity factors were also calculated using hydrodynamic chromatography (HDC; a method that differs from slalom chromatography due to the low shear rates). Selectivity factors were determined as described above. As shown in Table 5, in general, a particle size of 10 μm and a flow rate of >2.5 mL/min afforded good separation as measured by selectivity factors for nucleic acid molecules between 25,000 bp/nt and 100,000 bp/nt, including both between double-stranded species and between single-and double-stranded species.

TABLE 5
Specificity Factors Using a 10 μm Particle

| | | | Nucleic Acid Length | | | | | |
|---|---|---|---|---|---|---|---|---|
| Flow | Selectivity | | (kilonucleotides/kilobasepairs) | | | | | |
| Rate | Factor | Method | 1 | 5 | 10 | 25 | 50 | 100 |
| 2.5 | α_{ds/ss} | HDC | 0.97 | 0.93 | 0.90 | 0.87 | 0.85 | — |
| mL/min | | SC | 1.00 | 1.02 | 1.05 | 1.16 | 1.31 | 1.53 |
| | α_{ds2/ds1} | HDC | — | 0.94 | 0.96 | 0.93 | 0.95 | — |
| | | SC | — | 1.02 | 1.03 | 1.11 | 1.16 | 1.29 |
| 7.5 | α_{ds/ss} | HDC | 0.97 | 0.93 | 0.90 | 0.87 | 0.85 | — |
| mL/min | | SC | 1.00 | 1.04 | 1.08 | 1.19 | 1.3 | 1.40 |
| | α_{ds2/ds1} | HDC | — | 0.94 | 0.96 | 0.93 | 0.95 | — |
| | | SC | — | 1.03 | 1.04 | 1.13 | 1.20 | 1.33 |
| 15 | α_{ds/ss} | HDC | 0.97 | 0.93 | 0.90 | 0.87 | 0.85 | — |
| mL/min | | SC | 1.01 | 1.04 | 1.08 | 1.16 | 1.23 | 1.30 |
| | α_{ds2/ds1} | HDC | — | 0.94 | 0.96 | 0.93 | 0.95 | — |
| | | SC | — | 1.04 | 1.05 | 1.13 | 1.20 | 1.34 |

In sum, the presently disclosed methods are capable of separating nucleic acid molecules and mixtures thereof across a range of nucleotide lengths (ranging from 1,000 to 100,000 base pairs/nucleotides). In general, a larger particle size is able to separate larger nucleic acid molecules, and vice versa (e.g., smaller particle sizes are able to separate smaller nucleic acid molecules. The methods have further demonstrated the ability to separate both single-stranded and double-stranded molecules.

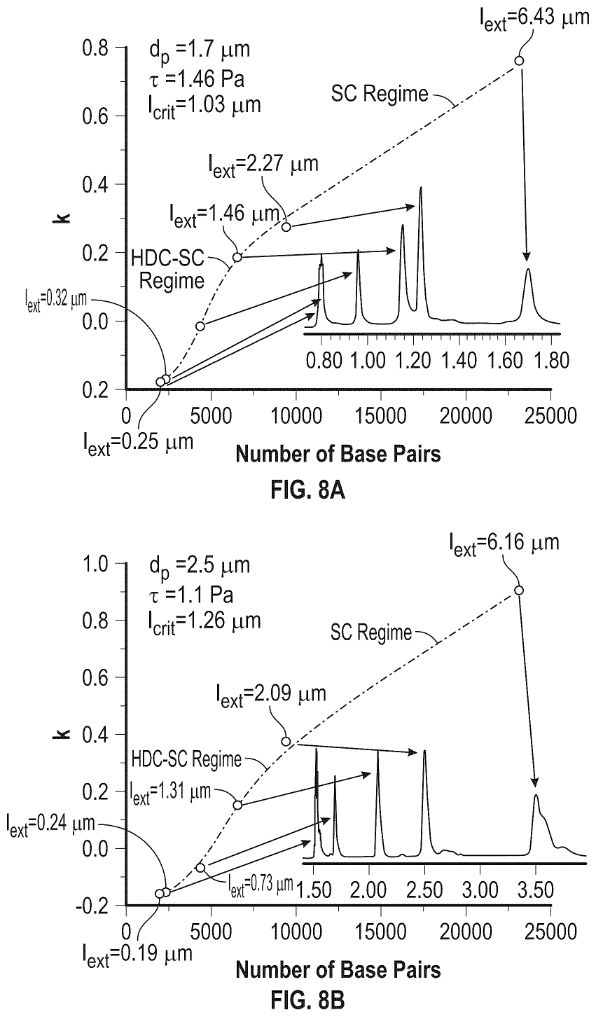

The methods described above further allow for the measurement of dsDNA length using known standards as depicted in FIG. 8A (for 1.7 μm particles) and FIG. 8B (for 2.5 μm particles). FIG. 8A-8B show the correlation between dsDNA length as measured by number of base pairs and the k coefficient.

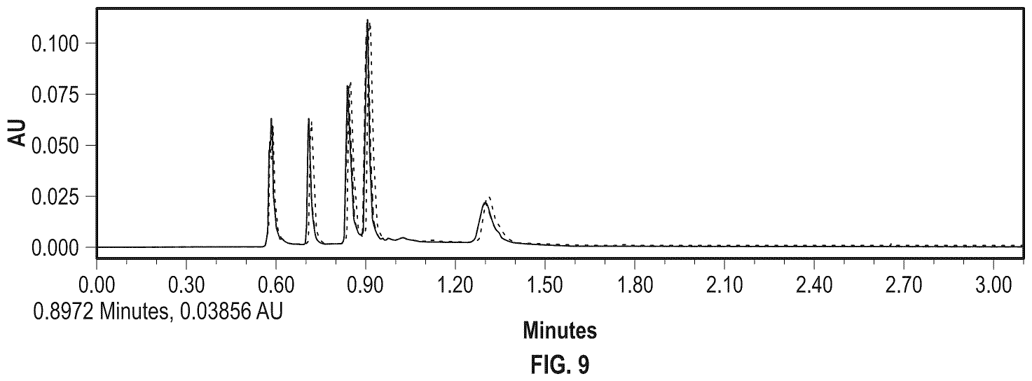

The methods described above further allow for the separation of DNA and RNA using 1.7 μm diethylene bridged hybrid particles (BEH) having a diol-bonded surface and an average pore size of 130 Å as shown in FIG. 9.

### Example 5: Effect of Ionic Strength on the Separation of Double-Stranded DNA

The effect of the ionic strength of the mobile phase on the separation of double-stranded DNA was assessed. A double-stranded DNA (dsDNA) plasmid digest was loaded onto a 4.6 mm×300 mm column comprising 2.6 μm nonporous silica particles and eluted using a mobile phase of 1× PBS (high ionic strength), 0.1× PBS, 0.01× PBS, or 0.001× PBS (low ionic strength). The column hardware was coated with a C2-PEG high-performance surface.

As shown in FIG. 5, decreasing the ionic strength of the mobile phase reduced retention time (e.g., peaks eluted faster) while improving peak resolution and k₁ values of said peaks. At 1× PBS, higher molecular weight peaks eluted past 10 minutes, with k₁ factors above 0.35. With the 2.6 μm nonporous silica particles used in this example, decreasing the ionic strength of the mobile phase from 1× PBS to either 0.1× PBS or, more preferably, 0.01× PBS, resulted in overall improved separation of the dsDNA sample. The results shown for 0.1× PBS and 0.01× PBS illustrate peaks eluted with k₁ factors at or below 0.35. Lowering the ionic strength to 0.001× PBS resulted in DNA melting due to instability of the sample in the mobile phase.

The ionic strength of the mobile phase may further be adjusted to improve peak shape and alter retention time, taking into consideration the sample being tested and the net negative charge of the particles used in the column. For example, adjustment or modification of the surfaces of particles can be controlled to result in a net negative charge of the particle surface, thus affecting the ionic strength conditions of the elution in the column.

### Example 6: Separation of Double-Stranded RNA (dsRNA) Species

A sample of double-stranded RNA (dsRNA) was loaded onto a 4.6 mm×300 mm column comprising diethylene bridged hybrid (BEH) diol particles and eluted using either a low shear flow rate (0.025 mL/min) or high shear flow rate (1.2 mL/min). The column hardware was coated with a C2-PEG high-performance surface. As shown in FIG. 6, increasing the flow rate from 0.025 mL/min to 1.2 mL/min shifted the retention of the dsRNA species, allowing for the separation of impurity conformers and the target, linear dsRNA in less than 1 minute.

### Example 7: Separation of Circular/Supercoiled and Linear DNA Species

A sample comprising either supercoiled DNA (ΦX 174 RF I DNA) or circular DNA (ΦX 174 RF II DNA) were loaded onto a 4.6 mm× 150 mm column comprising 1.7 μm ethylene bridged hybrid (BEH) particles having an average pore size of 45 Å. The column hardware was coated with a C2-PEG high-performance surface. Samples were flowed through the column using 100 mM phosphate buffer (pH 8) at 0.2 mL/min, 0.3 mL/min, 0.5 mL/min, or 1 mL/min. As shown in FIG. 7A-7B, increasing flow rate allowed for the separation of linear dsDNA impurities from the supercoiled DNA sample (FIG. 7A) and circular DNA sample (FIG. 7B), respectively.

### Example 8: Detection of Separated Nucleic Acids Using Multi-Angle Light Scattering (MALS)

A sample comprising a lambda DNA-HindIII digest (available from Promega Corporation) was loaded onto a 4.6 mm×300 mm column comprising 2.5 μm diethylene bridged hybrid (BEH) particles having an average pore size of 125 Å. The column hardware was coated with a C2-PEG high-performance surface. Sample was loaded at either a concentration of 5 μg or 0.5 μg and flowed through the column using 1× phosphate buffered saline (PBS) at a 0.2 mL/min flow rate. Eluent was detected using a multi-angle light scattering (MALS) detector. As shown in FIG. 10, the DNA digest could be separated and detected using MALS, particularly at the higher concentration with 0.5 μg representing the lower limit of detection for light scattering.

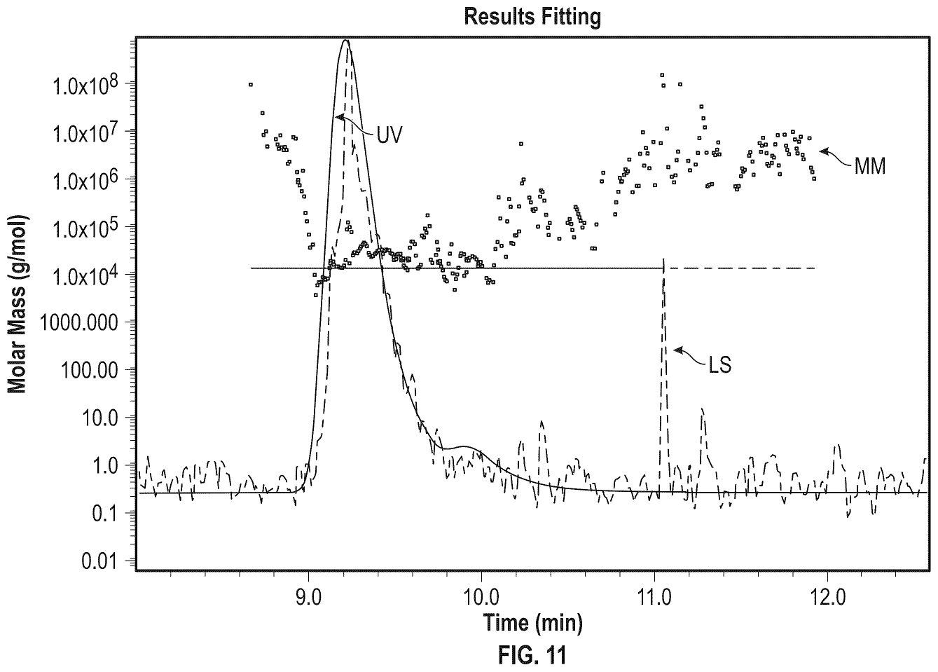

As a calibrant, a sample comprising a small interfering RNA (siRNA) was loaded onto a 4.6 mm×300 mm column comprising 2.5 μm diethylene bridged hybrid (BEH) particles having an average pore size of 125 Å. The column hardware was coated with a C2-PEG high-performance surface. Sample was loaded at a concentration of 1 μg and flowed through the column using 1× PBS at a 0.2 mL/min flow rate. Eluent was detected using a MALS detector. FIG. 11 provides a chromatograph of the sample detected using MALS. The trace labeled LS and UV represents the light scattering measurement and the ultraviolet measurement, respectively. The black squares represent the molar mass (MM) measurements. The expected monomer mass of the siRNA was 13.2 kDa and the observed, fitted monomer mass was ˜12.4 kDa. Thus, siRNA was determined to be a suitable calibrant for MALS detection in conjunction with the provided methods.

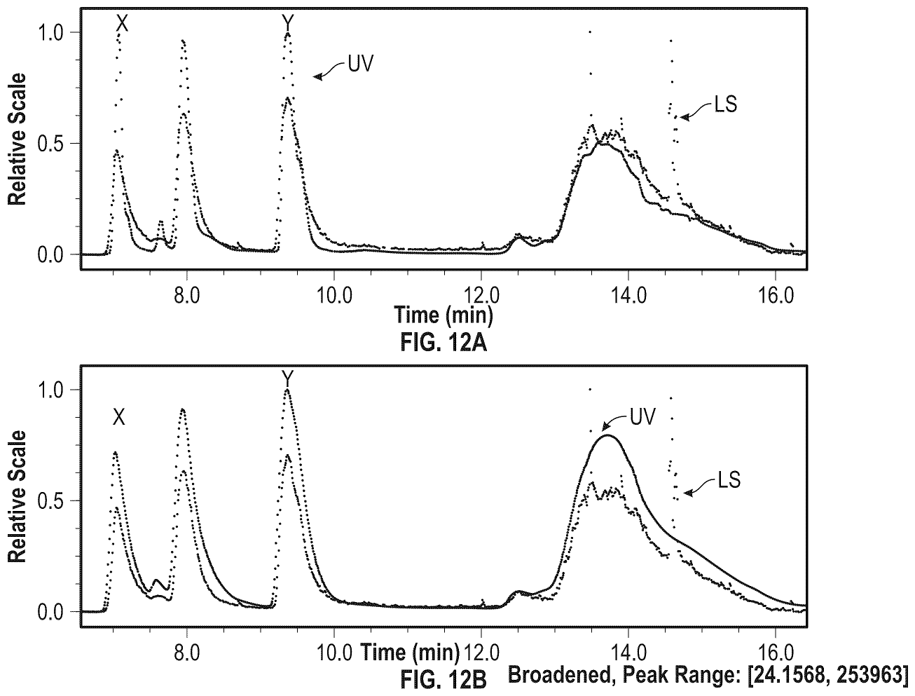

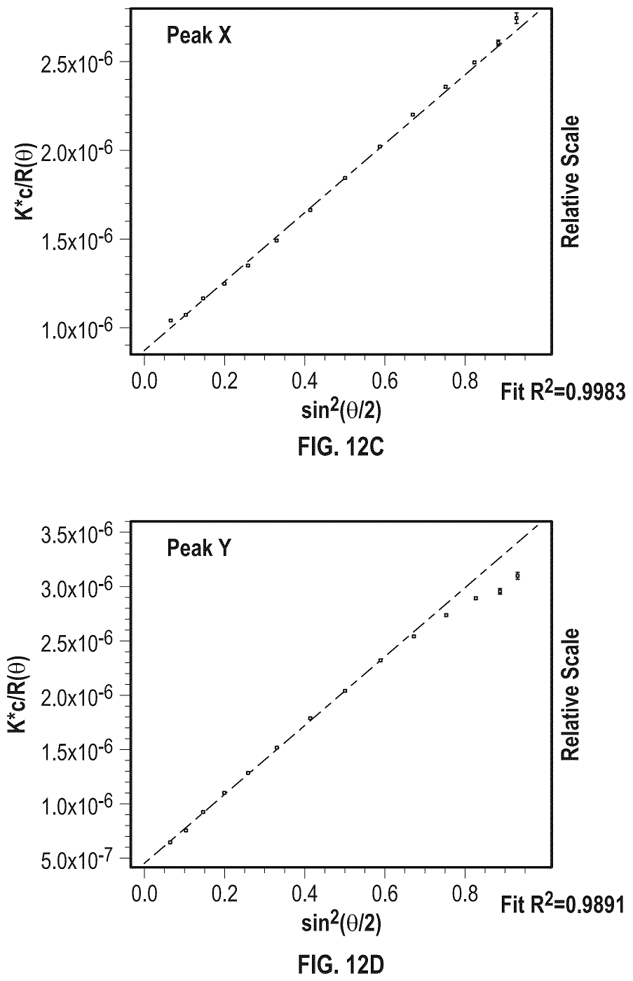

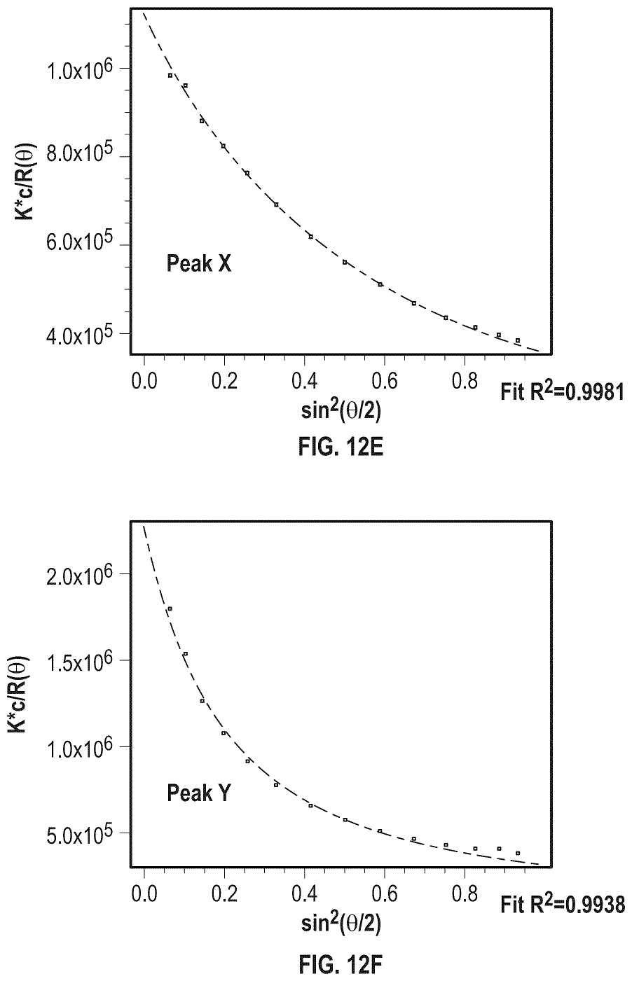

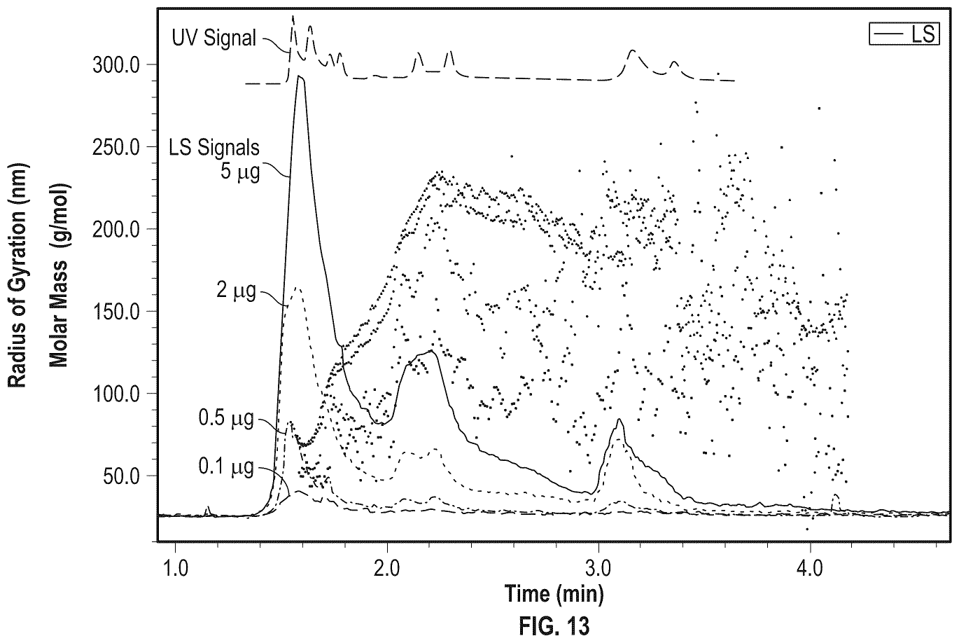

Using siRNA as the calibrant, the DNA-HindIII digest was modeled to determine molar mass. FIG. 12A-12B provide chromatographs of the DNA-HindIII digest before (FIG. 12A) and after (FIG. 12B) broadening of the UV signal. Small peaks (corresponding to lower molecular weight DNA fragments) fit to Zimm formalism as shown in FIG. 12C (Peak X) and FIG. 12D (Peak Y) and Random Coil as shown in FIG. 12E (Peak X) and FIG. 12F (Peak Y). FIGS. 12C and 12D provide the Zimm plots for peak X and Y as labeled in corresponding FIG. 12A-12B. FIGS. 12E and 12F provide the Random Coil plots for peak X and Y as labeled in corresponding FIG. 12A-12B. Methods of modeling MALS-derived data and determining relevant parameters for said models are known in the art and are described in, for example, U.S. Pat. No. 6,651,009, incorporated herein by reference. FIG. 13 demonstrates the light scattering and molar mass measurements for samples detected at 0.1 μg, 0.5 μg, 2 μg, and 5 μg.

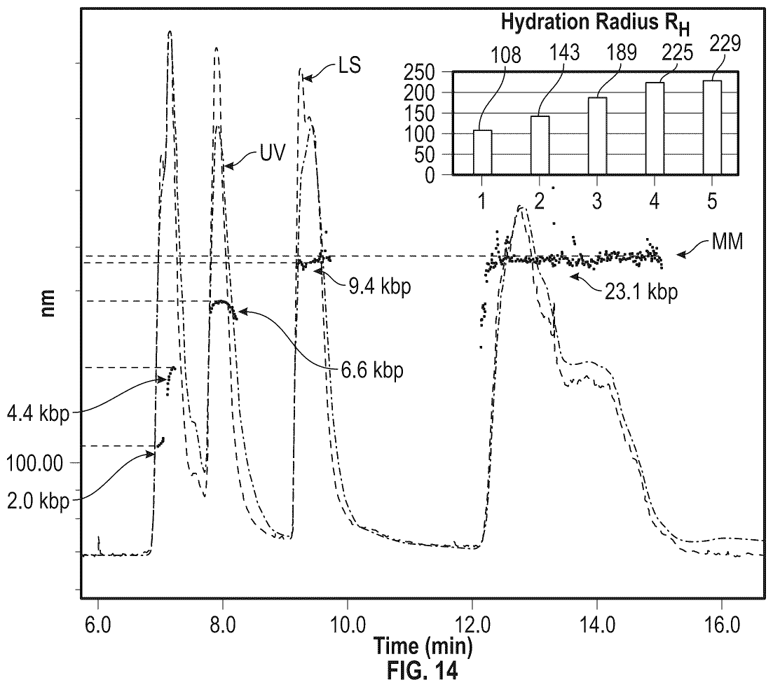

In addition to MALS detection, sample can be detected using dynamic light scattering (DLS) to determine the hydration radius (RH). Sample comprising the lambda DNA digest was loaded onto 4.6 mm×300 mm column comprising 2.5 μm diethylene bridged hybrid (BEH) particles having an average pore size of 125 Å. Sample was flowed through the column with a 0.2 mL/min flow rate and detected using MALS and DLS detectors. FIG. 14 provides a representative chromatograph of the detected nucleic acid species, including both UV and light scattering traces, molar mass measurements (dots), and hydration radius calculations (top right bar graph).

### Example 9: Separation of Nucleic Acids From Large Molecules

The methods of the present technology may be used to separate nucleic acids (such as dsDNA, ssDNA, dsRNA, or ssRNA) from additional large molecules, including biomolecules (proteins such as enzymes) and buffer components. Sample comprising a dsDNA ladder having DNA fragments of 2 kb, 4.4 kb, 6.6 kb, 9.4 kb, and 23.1 kb, RNAse, pyrophosphatase, NTPs, buffer, and T7 RNA polymerase was separated on a column comprising 2.5 μm diethylene bridged hybrid (BEH) particles having either 125 Å or 250 Å pore size. As shown in the top of FIG. 15, the pore size of 125 Å resulted in the co-elution of DNA with additional components present in the sample. In contrast and as shown in the bottom of FIG. 15, when the 250 Å pore size particle is used, DNA fragments ranging from 2 kb to 9.4 kb eluted from the column before the additional components present in the sample. Thus, the 250 Å pore size particle can be used with the provided methods to separate nucleic acids, such as DNA or RNA, from additional components present in a mixture.

### Example 10: Slalom Chromatography With Non-Porous Particles

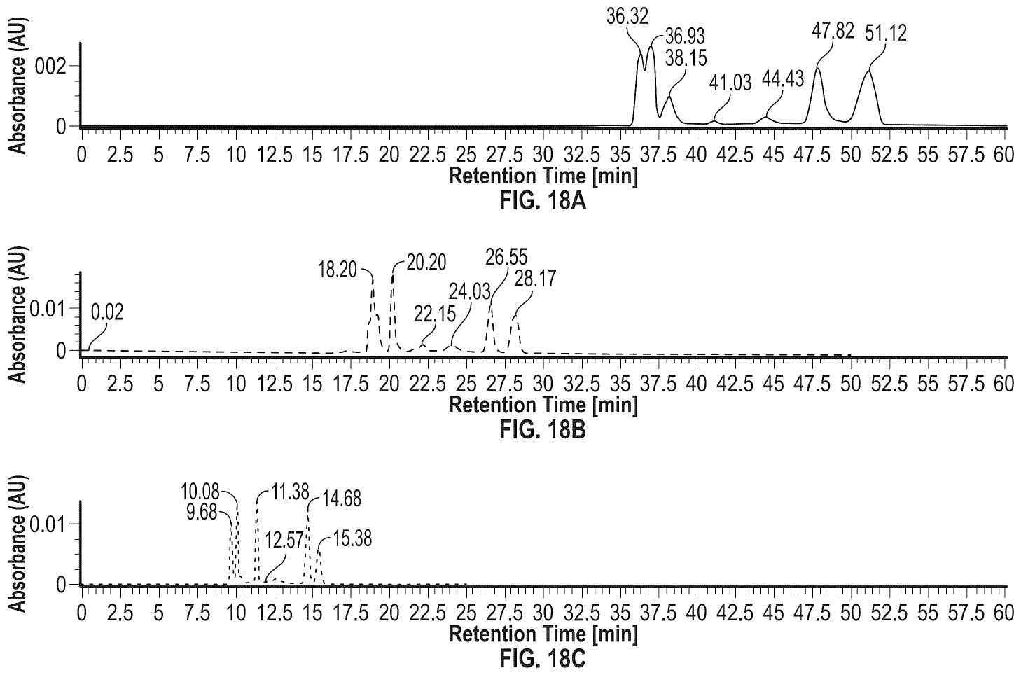

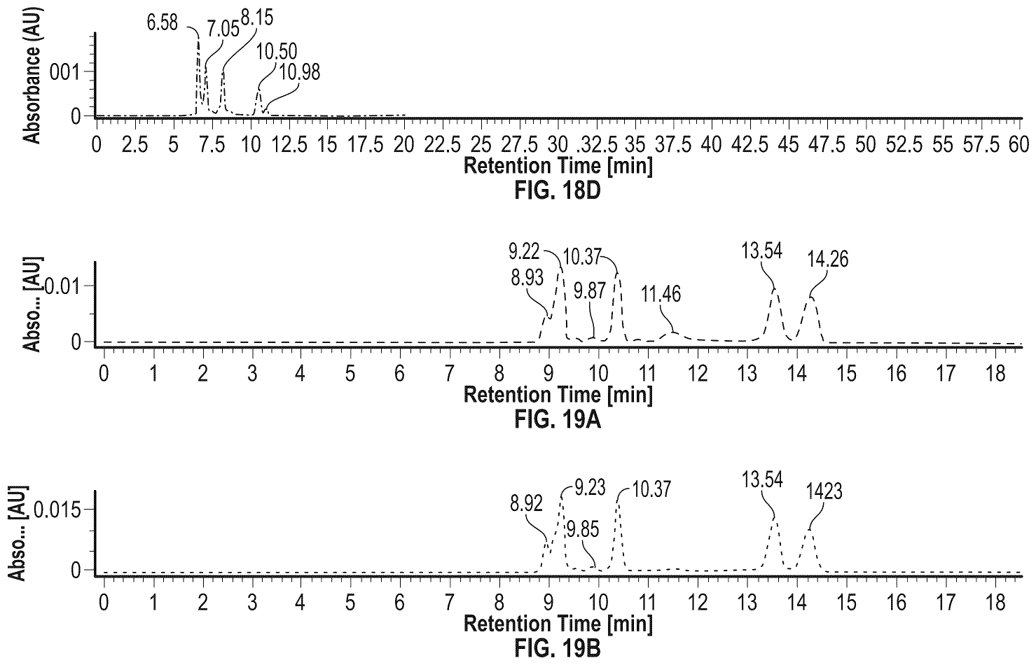

Slalom chromatography was performed using columns including non-porous silica based particles of varying sizes at varying flow rates. FIG. 18A-18D shows a chromatography experiment performed on a sample containing a λ-DNA-HindIII digest using a chromatography column comprising a 4.6 mm×300 mm column with 3 μm silica non-porous silica particles with a water mobile phase at a variety of flow rates. At a low-flow rate (0.05 mL/min), the experiment took nearly 52 total minutes. Many peaks are observed, however several peaks overlap with each other and are not distinct. By increasing the flow rate to 0.1 mL/min (FIG. 18B), 0.2 mL/min (FIG. 18C), or 0.3 mL/min (FIG. 18D), better resolution is observed between the major peaks in the spectrum. Moreover, the time to completely elute all components of the sample was drastically reduced.

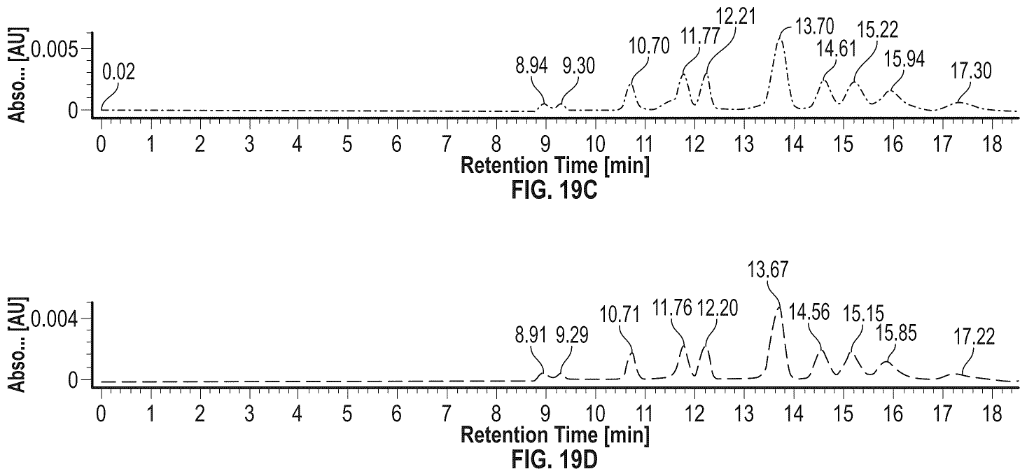

The experiment was then repeated using 2.6 um non-porous silica particles (FIG. 19A-D). Each experiment, was performed at pH of 7.4 with 1 M phosphate buffered saline as a mobile phase. In two experiments, the sample was a λ-DNA-HindIII digest (FIG. 19A and FIG. 19B). In the other two experiments, the sample was a λ-DNA Monocut Mix (FIG. 19C and FIG. 19D). Further, two experiments (FIG. 19B and FIG. 19D) included 0.5% by volume 2-[2-(diethylamino)ethoxy]ethanol as a mobile phase additive. Good separation was observed in each experiment.

### Example 11: Effects of pH in Slalom Chromatography Using Non-Porous Particles

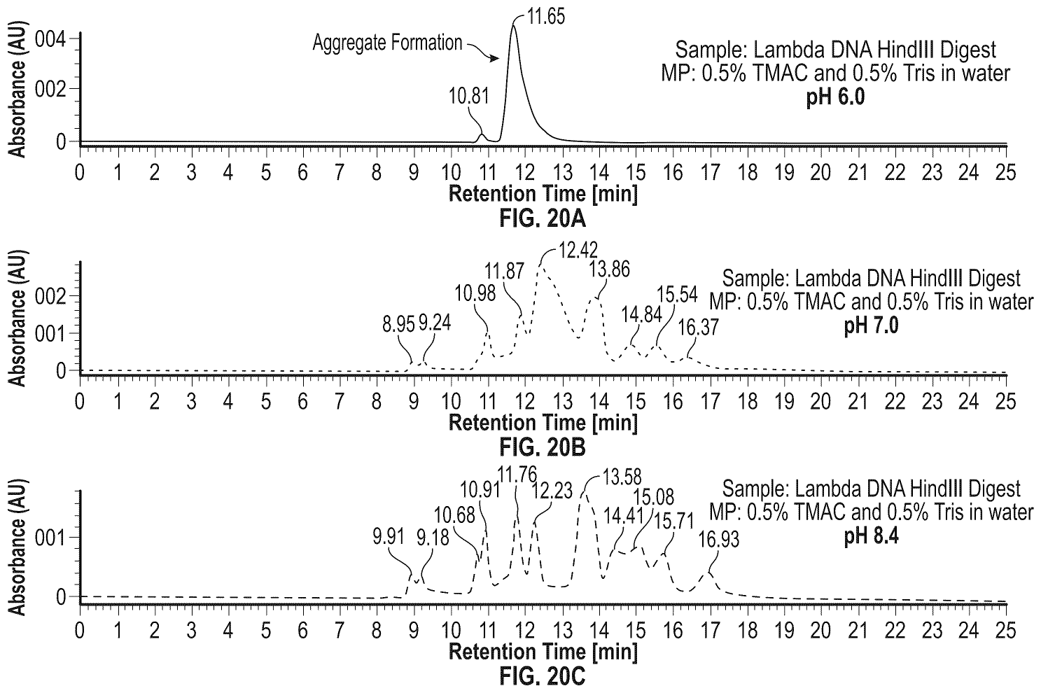

A 4.6×150 mm chromatography column including 2.6 μm non-porous silica particles was then examined at variable pH (FIG. 20A-C). In each experiment, the sample was λ-DNA-HindIII digest, the mobile phase was water including 0.5% by volume tetramethyl ammonium chloride and 0.5% by volume trismethyl aminomethane, and the flow rate was 0.1 mL/min. At an acidic pH (FIG. 20A), only two peaks were observable, due in part to aggregate formation, with the aggregate eluting as a single peak at approximately 11.65 min. At a neutral pH (FIG. 20B), the aggregate was better resolved as a plurality of overlapping peaks. At a basic pH (FIG. 20C), the entire chromatogram was better resolved, with multiple distinct peaks capable of being resolved. This experiment indicates that slalom chromatography of oligonucleotides is particularly effective at basic pH.

### Example 12: Effect of Ionic Strength on Separation With Non-Porous Particles

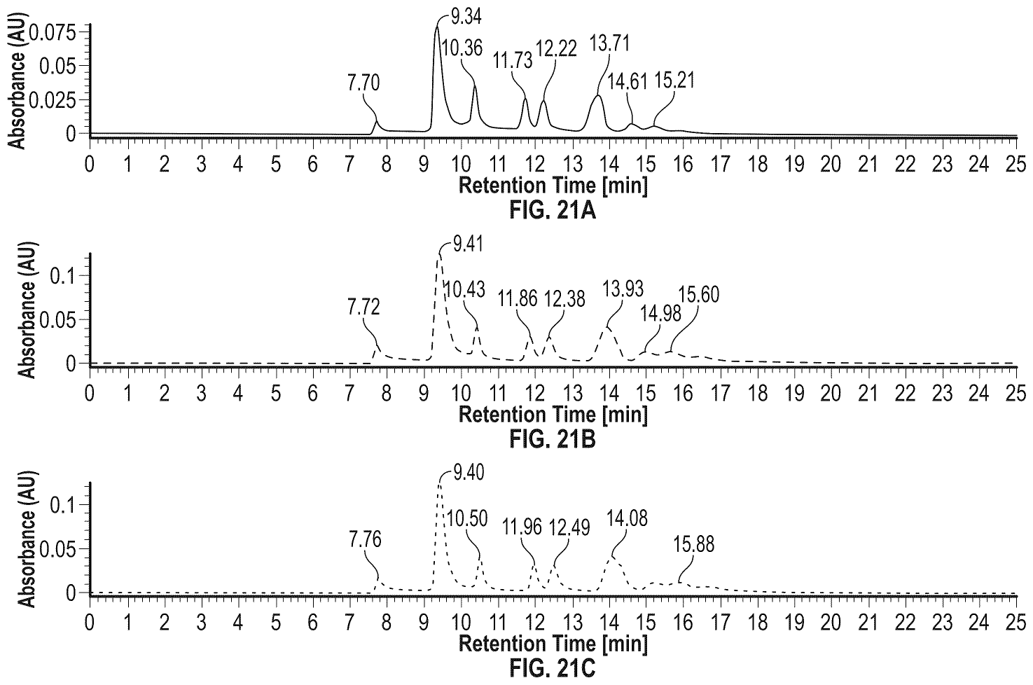

The impact of the ionic strength of the solution in a separation using non-porous particles was then examined. A λ-DNA Monocut Mix sample was investigated using a 2.1×300 mm column including 2.2 μm BEH particles was investigated using a flow rate 0.05, a pH of 7.4, and three different concentrations of buffer. FIG. 21A shows the chromatogram at 1× PBS. FIG. 21B shows the chromatogram at 2× PBS. FIG. 21C shows the chromatogram at 4× PBS. Good separation was observed in each experiment.

### Example 13: Slalom Chromatography With Superficially Porous Particles

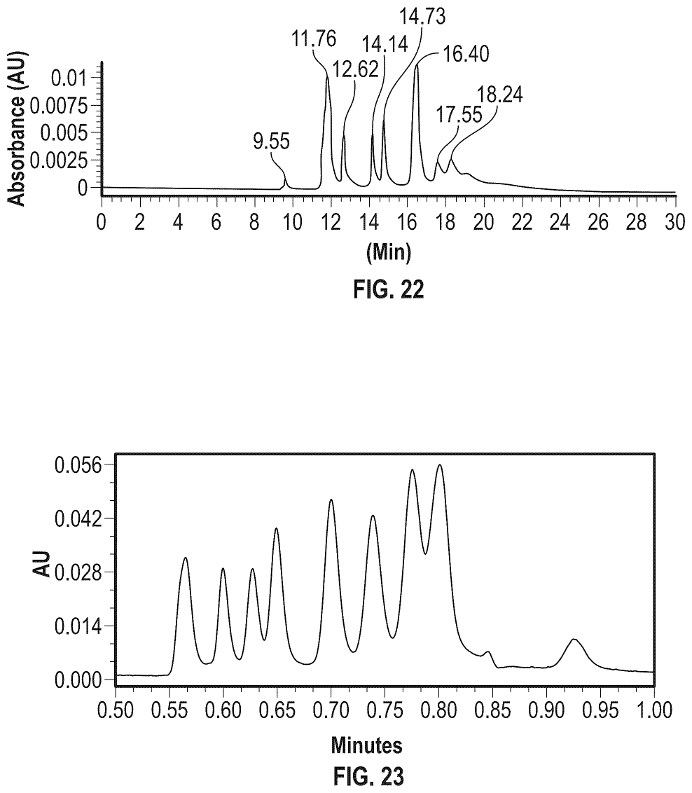

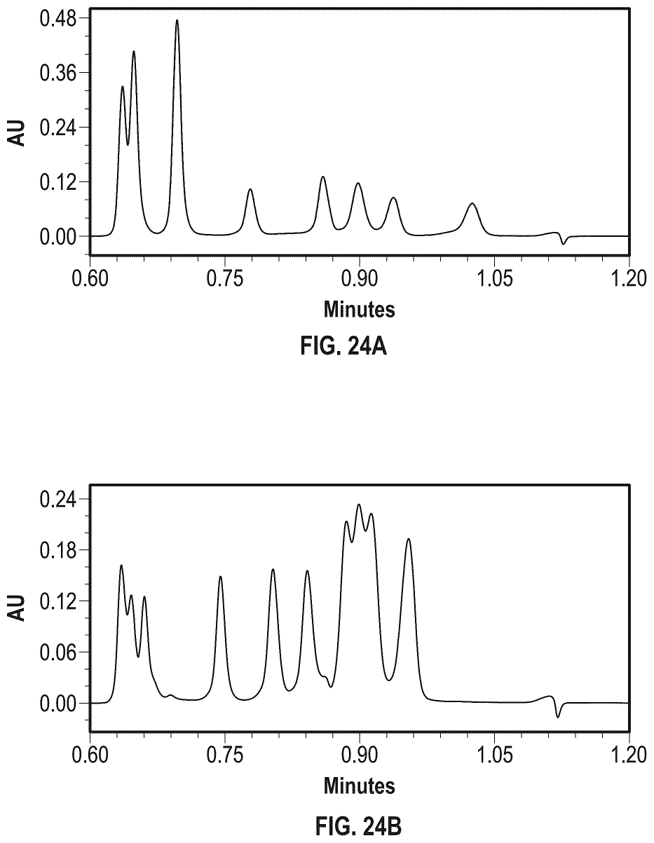

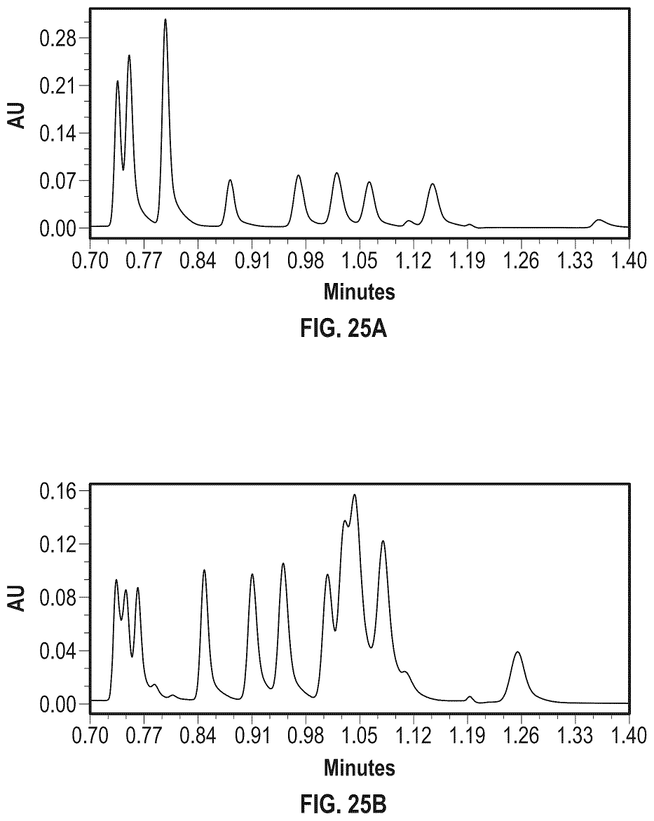

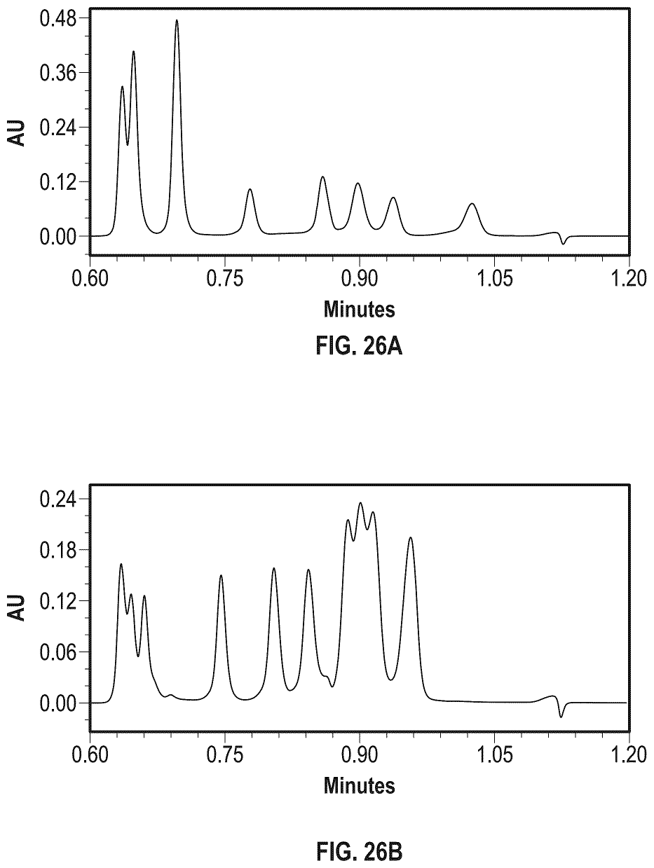

Slalom chromatography of oligonucleotides using a variety of superficially porous particles was investigated (FIG. 22-FIG. 26). Particle types include 3.0 μm hybrid (inorganic-organic) particles having a solid core (such as the particles available from Waters Corporation sold in connection with the CORTECS trade name) (FIG. 22); 2.7 μm silica solid-core particles (e.g., CORTECS silica particles) (FIG. 23), 1.6 μm solid-core silica particles with T3 bonding (e.g., Cortecs-T3 available from Waters Corporation) (FIG. 24A-24B), 1.6 μm solid-core silica particles having 90 Å average pore size (e.g., CORTECS 90 Å) (FIG. 25A-25B), and 1.6 μm solid-core silica particles having an average pore size of 120 Å (e.g., CORTECS 120 Å) (FIG. 26A-26B). Samples included λ-DNA Monocut Mix (FIG. 22), λ-DNA BstEII digest (FIG. 23, FIG. 24B, FIG. 25B, FIG. 26B), or a 1 kilobasepair DNA ladder (FIG. 24A, FIG. 24B, FIG. 25B). The experiments with 1.6 μm silica-core with T3 bonding (e.g., Cortecs-T3) further included 20% acetonitrile in the mobile phase. Good separation was observed in each experiment.

### Example 14: Slalom Chromatography Using Diol Functionalized Particles

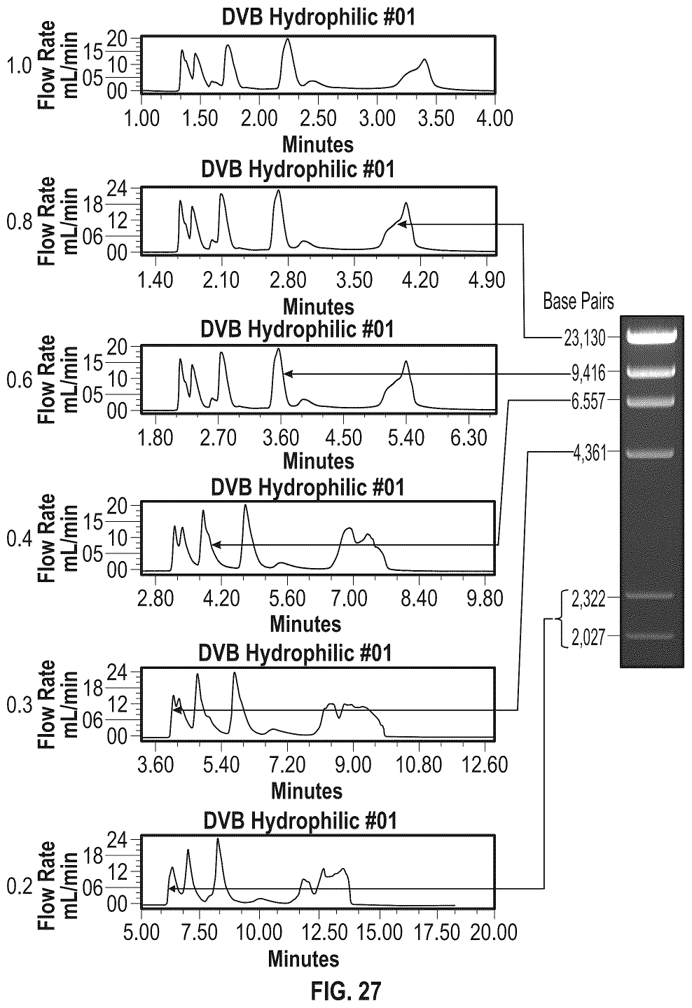

Slalom chromatography of oligonucleotides using particles including a diol functionalized surface was investigated. FIG. 27 shows the separation of a λ-DNA HindiII digest sample using a 4.6×300 mm column including non-porous 3.5 μm divinylbenzene (DVB) organic polymer particles which were surface modified using ethylene glycol. The mobile phase was phosphate at pH 8. Good separation was observed across flow rate. Different oligonucleotides were better resolved at different flow rates.

### Example 15: Slalom Chromatography Using Sulfate Functionalized Particles

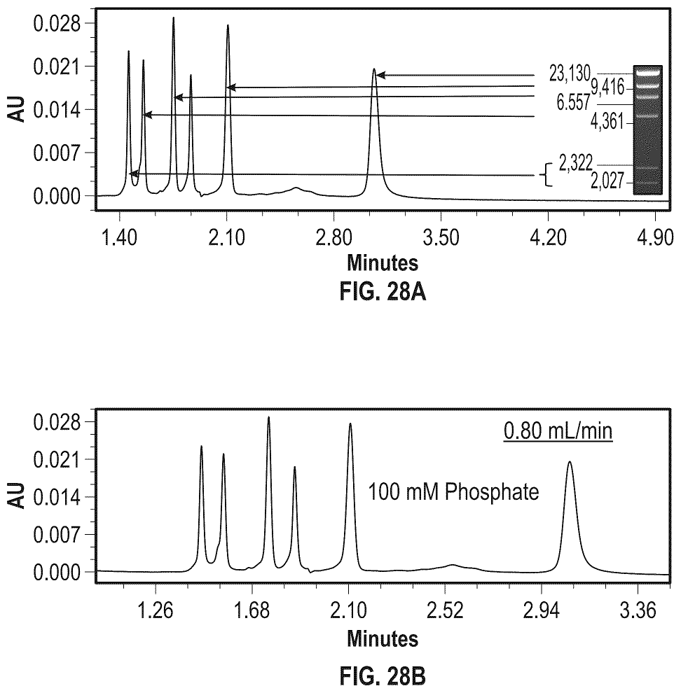

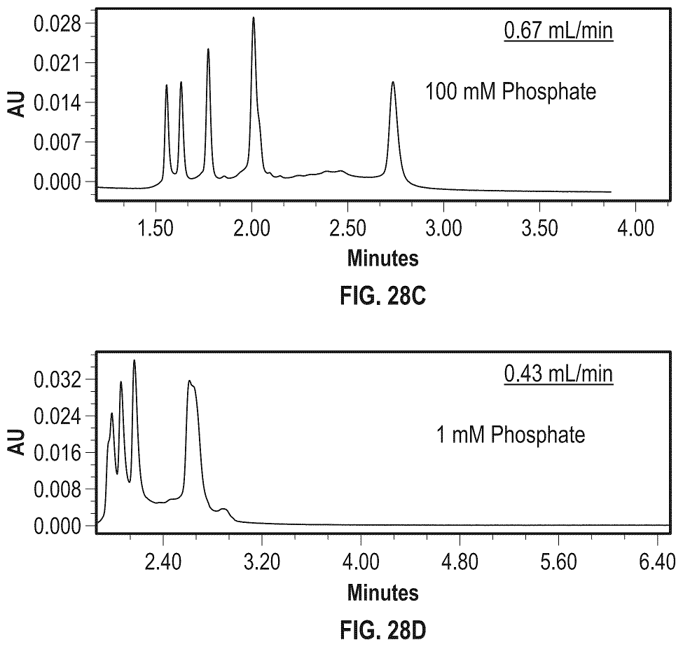

Slalom chromatography of oligonucleotides using particles including a sulfate functionalized surface was investigated. FIG. 28A-28C show the separation efficiency of the particles described in example 14 after further functionalization to include a SO₃⁻ group. The particles were loaded into 4.6×300 mm column with a mobile phase of 100 mm phosphate at pH 8. A flow rate of 0.8 mL/min was used in the initial investigation. The sulfate functionalized particles displayed significantly narrowed peaks compared to the unfunctionalized particles (FIG. 28A). This experiment indicates that particles functionalized with negatively charged surface groups show particular effect at separating DNA. FIG. 28B-28D demonstrate the ionic strength and flow rate dependence of these SO₃⁻ functionalized particles is similar to that observed in similar experiments.

### Example 16: Slalom Chromatography Using Amide Functionalized Particles

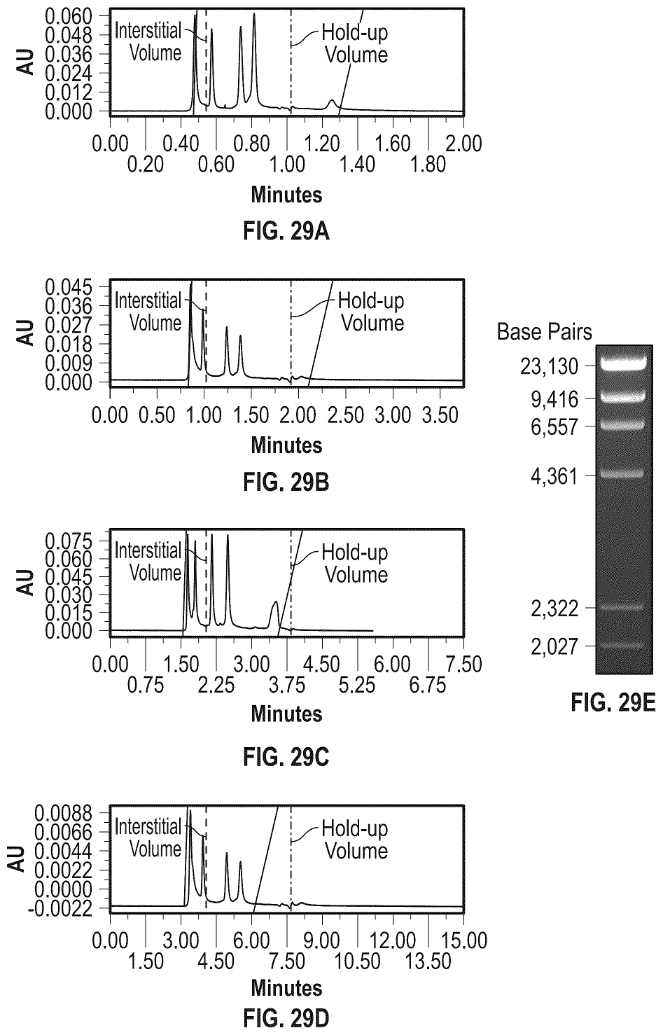

Slalom chromatography of oligonucleotides using particles including an amide functionalized surface was investigated. FIG. 29A-29D shows the separation efficiency of a 2.1×150 mm column including 1.7 μm BEH130 particles functionalized to include an amide surface. The particles were investigated at a variety of flow rates (0.375 mL/min (FIG. 29A), 0.20 mL/min (FIG. 29B), 0.10 mL/min (FIG. 29C), and 0.05 mL/min (FIG. 29D). FIG. 29E, then, shows the gel electrophoresis separation of the sample indicating the possible oligonucleotide species which the experiment may resolve. Good separation was observed in each experiment. These experiments indicates that reverse phase liquid chromatography (RPLC) methods are compatible with slalom chromatography methods disclosed herein.

### Example 17: Slalom Chromatography Using Particles with a Hydrophobic Surface

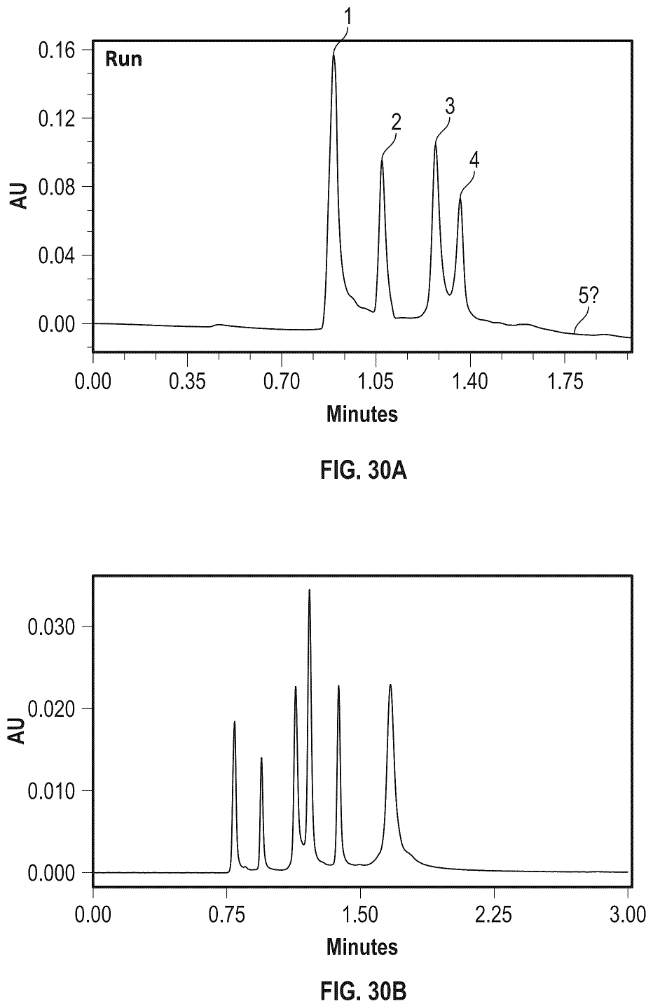

Slalom chromatography of oligonucleotides using particles including a particle surface functionalized with a hydrophobic group was investigated. 1.7 μm BEH particles having an average pore size of 45 Å and functionalized with a trimethylchlorosilane surface group were loaded into a 4.6×300 mm column. A separation was using a mobile phase of 100 mM phosphate at pH 8 in a 70% water, 30% acetonitrile by volume solution with a flow rate of 0.9 mL/min (FIG. 30A). Separation results were compared to an unfunctionalized surface (FIG. 30B) at a flow rate of 1.0 mL/min. Due to differences in flow pressure resistance, a flow rate of 0.9 mL/min (as used in the functionalized surface experiment) and a flow rate of 1.0 mL/min (as used in the unfunctionalized surface experiment) resulted in a similar pressure drops across the column length in both experiments.

Unlike the hydrophilic surfaces used in Examples 14-16, the hydrophobic C1 surface displayed broader peaks that were less resolved compared to an unfunctionalized surface.

1.-9. (canceled)

10. A method of separating RNA, the method comprising:
a) loading a sample comprising a plurality of single-stranded RNA (ssRNA) molecules and a plurality of double-stranded RNA (dsRNA) molecules onto a chromatographic column, wherein the column comprises a plurality of particles;
b) eluting the sample from the column, wherein the eluting results in a zone retention factor (k1) of less than 0.35; and
c) detecting the plurality of ssRNA molecules and/or the plurality of dsRNA molecules in the eluent.

11. (canceled)

12. The method of claim 10, wherein the eluting is performed in under 10 minutes.

13. The method of claim 10, wherein step b) is performed with an efficiency (N)>40,000.

14. (canceled)

15. The method of claim 10, wherein the ssRNA and/or the dsRNA comprise between about 1,000 base pairs to about 100,000 base pairs.

16.-20. (canceled)

21. The method of claim 10 , wherein step c) is performed using an ultraviolet (UV) detector, a tunable ultraviolet (TUV) detector, a multi-angle light scattering (MALS) detector, or a dynamic light scattering (DLS) detector.

22. The method of claim 10 wherein step b) is performed using a flow rate of greater than 0.5 mL/min and wherein the dsDNA, the ssDNA, the ssRNA and/or the dsRNA comprise between about 2,000 base pairs to about 25,000 base pairs.

23. (canceled)

24. The method of claim 10, wherein step b) is performed using a flow rate of greater than 0.75 mL/min and wherein the dsDNA, the ssDNA, the ssRNA and/or the dsRNA comprise between about 5,000 base pairs to about 50,000 base pairs.

25. (canceled)

26. The method of claim 10, wherein step b) is performed using a flow rate of greater than 1.0 mL/min and wherein the dsDNA, the ssDNA, the ssRNA and/or the dsRNA comprise between about 25,000 base pairs to about 50,000 base pairs.

27. (canceled)

28. The method of claim 10, wherein step b) is performed using a flow rate of greater than 2.5 mL/min and wherein the dsDNA, the ssDNA, the ssRNA and/or the dsRNA comprise between about 25,000 base pairs to about 100,000 base pairs.

29.-31. (canceled)

32. The method of claim 10, wherein the plurality of particles comprise a plurality of non-porous particles, a plurality of porous particles, or a plurality of superficially porous particles.

33.-34. (canceled)

35. The method of claim 32, wherein the plurality of porous particles or the plurality of superficially porous particles have an average pore diameter of between 20 Å to 900 Å.

36.-39. (canceled)

40. The method of claim 10, wherein the plurality of particles comprise an inorganic material.

41. (canceled)

42. The method of claim 10, wherein the plurality of particles comprise an organic polymer.

43. (canceled)

44. The method of claim 10, wherein the plurality of particles comprise a hybrid organic-inorganic material.

45. The method of claim 44, wherein the hybrid organic-inorganic material comprises a bridged ethylene hybrid (BEH) material.

46. The method of claim 10, wherein the plurality of particles comprise a functional group bound to the surface of the particle, wherein the functional group is a hydrophilic functional group.

47.-50. (canceled)

51. The method of claim 10, wherein the plurality of particles comprise a functional group bound to the surface of the particle, wherein the functional group is a hydrophobic functional group.

52. A method of separating RNA, the method comprising:
a) loading a sample comprising a plurality of single-stranded RNA (ssRNA) molecules and a plurality of double-stranded RNA (dsRNA) molecules onto a chromatographic column, wherein the column comprises a plurality of particles;
b) eluting the sample from the column, wherein the eluting is performed in at most 3 minutes; and
c) detecting the plurality of ssRNA molecules and/or the plurality of dsRNA molecules in the eluent.

53. The method of claim 52, wherein the eluting is performed in at most 1 minute.

54. The method of claim 52, wherein the plurality of particles comprise bridged ethylene hybrid (BEH) particles comprising a pore size of about 45 Å.

55. The method of claim 52, wherein the eluting of step (b) comprises a flow rate of about 1 mL/min.
